(19) United States
(12) Patent Application Publication
(10) Pub. No.: US 2011/0257171 A1
(43) Pub. Date: Oct. 20, 2011

(54) NOVEL IMIDAZO[1,2-A]PYRIDINE DERIVATIVES, METHOD FOR THE PREPARATION THEREOF, USE THEREOF AS MEDICAMENTS, PHARMACEUTICAL COMPOSITIONS AND NOVEL USE IN PARTICULAR AS MET INHIBITORS

(75) Inventors: Dominique Damour, Paris (FR); Conception Nemecek, Paris (FR); Patrick Nemecek, Paris (FR); Sylvie Wentzler, Paris (FR)

(73) Assignee: SANOFI-AVENTIS, Paris (FR)

(21) Appl. No.: 13/003,604

(22) PCT Filed: Jul. 16, 2009
(86) PCT No.: PCT/FR2009/051407

§ 371 Date: Jun. 30, 2011

(30) Foreign Application Priority Data
Jul. 18, 2008 (FR) 08/04085
May 19, 2009 (FR) 0902409

(51) Int. Cl. A61K 31/437; A61P 9/00; A61P 7/04; A61P 3/00; A61P 37/08; A61P 11/06; A61P 7/02; A61P 25/00; A61P 9/10; A61P 17/06; A61P 19/02; A61P 3/10; A61P 21/00; A61P 35/00; A61P 35/04; C07D 413/14; A61K 31/5377; C07D 471/04

(52) U.S. Cl. 514/233.2; 546/121; 514/300; 544/127

(57) ABSTRACT

The invention relates to the novel products of formula (I): in which: Ra is H, Hal, aryl or heteroaryl, which are optionally substituted; Rb is H, Rc, —COORc-CO-Rc or —CO—NRcRd; where Rc is alkyl, cycloalkyl, heterocycloalkyl, aryl and heteroaryl, all optionally substituted; Rd is H, alk or cycloalkyl; these products being in all the isomer forms and the salts, as medicaments, in particular as MET inhibitors.

The present invention relates to novel imidazo[1,2-a]pyridine derivatives, to the process for the preparation thereof, to the novel intermediates obtained, to the use thereof as medicaments, to the pharmaceutical compositions containing them and to the novel use of such imidazo[1,2-a]pyridine derivatives.

The present invention relates more particularly to novel imidazo[1,2-a]pyridine derivatives having an anticancer activity, via the modulation of the activity of proteins, in particular kinases.

To date, most of the commercially available compounds used in chemotherapy are cytotoxic agents which pose considerable problems in terms of side effects and tolerance by patients. These effects could be limited if the medicaments used acted selectively on cancer cells, to the exclusion of healthy cells. One of the solutions for limiting the adverse effects of a chemotherapy may thus consist in using medicaments that act on metabolic pathways or constituent elements of these pathways, predominantly expressed in cancer cells, and sparingly expressed or not expressed in healthy cells. The protein kinases are a family of enzymes that catalyse the phosphorylation of hydroxyl groups of specific residues of proteins, such as tyrosine, serine or threonine residues. Such phosphorylations can largely modify the function of proteins: thus, protein kinases play an important role in the regulation of a large variety of cell processes, including in particular metabolism, cell proliferation, cell adhesion and motility, cell differentiation or cell survival, certain protein kinases playing a central role in the initiation, development and accomplishment of cell cycle events.

Among the various cellular functions in which the activity of a protein kinase is involved, certain processes represent attractive targets for treating certain diseases. As an example, mention may in particular be made of angiogenesis and the control of the cell cycle and also that of cell proliferation, in which protein kinases can play a central role. These processes are in particular essential for the growth of solid tumours and also for other diseases: in particular, molecules that inhibit such kinases are capable of limiting unwanted cell proliferations such as those observed in cancers, and may play a part in preventing, regulating or treating neurodegenerative diseases such as Alzheimer's disease or neuronal apoptosis.

A subject of the present invention is novel derivatives with inhibitory effects on protein kinases. The products according to the present invention may thus in particular be used for preventing or treating diseases that may be modulated by inhibition of protein kinases.

The products according to the present invention in particular show anticancer activity, via the modulation of the activity of kinases. Among the kinases for which a modulation of the activity is sought, MET and also mutants of the MET protein are preferred.

The present invention also relates to the use of said derivatives for the preparation of a medicament for use in human therapy.

Thus, one of the objects of the present invention is to provide compositions that have an anticancer activity, by acting in particular on kinases. Among the kinases for which a modulation of the activity is sought, MET is preferred.

In the pharmacological section hereinafter, it is shown, in biochemical tests and on cell lines, that the products of the present application thus inhibit in particular the autophosphorylation activity of MET and the proliferation of cells whose growth depends on MET or on mutant forms thereof.

MET, or Hepatocyte Growth Factor Receptor, is a receptor with tyrosine kinase activity, expressed in particular by epithelial and endothelial cells. HGF, Hepatocyte Growth Factor, is described as the specific ligand of MET. HGF is secreted by the mesenchymal cells and activates the MET receptor, which homodimerizes. Consequently, the receptor autophosphorylates on the tyrosines of the catalytic domain Y1230, Y1234 and Y1235.

Stimulation of MET with HGF induces cell proliferation, scattering (or dispersion) and motility, resistance to apoptosis, invasion and angiogenesis.

MET and likewise HGF are found to be overexpressed in many human tumours and a wide variety of cancers. MET is also found to be amplified in gastric tumours and glioblastomas. Many point mutations of the MET gene have also been described in tumours, in particular in the kinase domain, but also in the juxtamembrane domain and the SEMA domain. Overexpression, amplification or mutations cause constitutive activation of the receptor and dysregulation of its functions.

The present invention thus relates in particular to novel inhibitors of the MET protein kinase and of its mutants, that can be used for an antiproliferative and antimetastatic treatment, in particular in oncology.

The present invention also relates to novel inhibitors of the MET protein kinase and of its mutants, that can be used for an anti-angiogenic treatment, in particular in oncology.

A subject of the present invention is the products of formula (I):

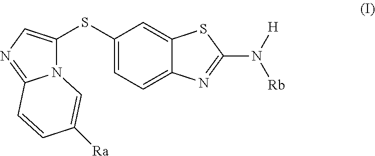

in which:

Ra represents a hydrogen atom; a halogen atom; an aryl radical; or a heteroaryl radical, these aryl and heteroaryl radicals being optionally substituted as indicated hereinafter;

Rb represents a hydrogen atom, an Rc, —COORc or —CO-Rc radical or a —CO—NRcRd radical;

where Rc represents an alkyl, cycloalkyl, heterocycloalkyl, aryl or heteroaryl radical, all these radicals being optionally substituted as indicated hereinafter;

Rd represents a hydrogen atom or an alkyl or cycloalkyl radical;

all the alkyl, cycloalkyl, heterocycloalkyl, aryl and heteroaryl radicals defined above being optionally substituted with one or more radicals chosen from halogen atoms, and the radicals: hydroxyl, alkoxy, CN, CF3, —NR1R2, —COOH, —COOalk, —CONR1R2, —NR1COR2, COR1, oxo and heterocycloalkyl, itself optionally substituted with one or more radicals chosen from halogen atoms, and hydroxyl, alkoxy, alkyl, CN, CF3, —NR3R4, COOH, —COOalk, —CONR3R4, —NR3COR4, —COR3 and oxo radicals;

the alkyl and cycloalkyl radicals also being optionally substituted with an aryl or heteroaryl radical, themselves optionally substituted with one or more radicals chosen from halogen atoms and hydroxyl, alkyl, alkoxy and NR3R4 radicals;

the cycloalkyl, heterocycloalkyl, aryl or heteroaryl radicals also being optionally substituted with an alkyl radical, itself optionally substituted with one or more radicals chosen from halogen atoms and hydroxyl, alkyl, alkoxy and NR3R4 radicals;

NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

the cyclic radicals that R1 and R2 or R3 and R4, respectively, can form, with the nitrogen atom to which they are attached, being optionally substituted with one or more radicals, which may be identical or different, chosen from halogen atoms, hydroxyl, oxo, alkoxy, NH₂, NHalk and N(alk)₂ radicals, and alkyl, phenyl, CH₂-phenyl and heteroaryl radicals, such that, in the latter radicals, the alkyl, phenyl and heteroaryl radicals are themselves optionally substituted with one or more radicals chosen from halogen atoms and the radicals: hydroxyl, alkyl and alkoxy containing from 1 to 4 carbon atoms, NH₂, NHalk and N(alk)₂;

all the alkyl (alk) and alkoxy radicals above containing from 1 to 6 carbon atoms;

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above, in which:

Ra represents a hydrogen atom; a halogen atom; or an aryl or heteroaryl radical, these aryl and heteroaryl radicals being optionally substituted as indicated hereinafter;

Rb represents a hydrogen atom, a —CO-Rc radical or a —CO—NRcRd radical;

where Rc represents an alkyl radical or a cycloalkyl radical, both optionally substituted with one or more radicals chosen from the radicals: hydroxyl, alkoxy, NR1R2, heterocycloalkyl, aryl and heteroaryl, themselves optionally substituted as indicated hereinafter;

Rd represents a hydrogen atom or an alkyl radical;

all the alkyl, cycloalkyl, heterocycloalkyl, aryl and heteroaryl radicals defined above being optionally substituted with one or more radicals chosen from halogen atoms, and the radicals: hydroxyl, alkoxy, —NR1R2, —COOH, —COOalk, —CONR1R2, alkyl and heterocycloalkyl itself optionally substituted with one or more radicals chosen from halogen atoms, and alkyl, COOH, —COOalk and —CONR3R4 radicals;

NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

the cyclic radicals that R1 and R2 or R3 and R4, respectively, can form, with the nitrogen atom to which they are attached, being optionally substituted with one or more radicals, which may be identical or different, chosen from halogen atoms, hydroxyl and alkoxy radicals, and alkyl, phenyl and CH₂-phenyl radicals, in which the alkyl or phenyl radicals are themselves optionally substituted with one or more radicals, which may be identical or different, chosen from halogen atoms and alkyl, hydroxyl, alkoxy, NH₂, NHalk and N(alk)₂ radicals;

all the alkyl (alk) or alkoxy radicals above containing from 1 to 6 carbon atoms,

said products of formula (I) being in all the possible racemic, enantiomeric and diastereoisomeric isomer forms, and also the addition salts with inorganic and organic acids or with inorganic and organic bases of said products of formula (I).

A subject of the present invention is thus the products of formula (I):

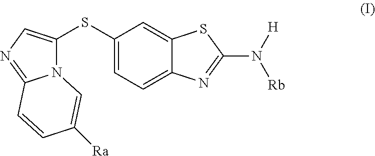

in which:

Ra represents a hydrogen atom; a halogen atom; an aryl radical; or a heteroaryl radical, these aryl and heteroaryl radicals being optionally substituted as indicated hereinafter;

Rb represents a hydrogen atom, an Rc, —COORc or —CO-Rc radical or a —CO—NRcRd radical;

where Rc represents an alkyl, cycloalkyl, heterocycloalkyl, aryl or heteroaryl radical, all these radicals being optionally substituted as indicated hereinafter;

Rd represents a hydrogen atom or an alkyl or cycloalkyl radical;

all the alkyl, cycloalkyl, heterocycloalkyl, aryl and heteroaryl radicals defined above being optionally substituted with one or more radicals chosen from halogen atoms, and hydroxyl, alkoxy, CN, CF₃, —NR1R2, —COOH, —COOalk, —CONR1R2 and —NR1COR2 radicals;

the alkyl radicals also being optionally substituted with an aryl or heteroaryl radical, themselves optionally substituted with one or more radicals chosen from halogen atoms and hydroxyl, alkyl, alkoxy and NR3R4 radicals;

the cycloalkyl, heterocycloalkyl, aryl or heteroaryl radicals also being optionally substituted with an alkyl radical, itself optionally substituted with one or more radicals chosen from halogen atoms and hydroxyl, alkyl, alkoxy and NR3R4 radicals;

NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

the cyclic radicals that R1 and R2 or R3 and R4, respectively, can form, with the nitrogen atom to which they are attached, being optionally substituted with one or more radicals, which may be identical or different, chosen from halogen atoms, hydroxyl, oxo, alkoxy, NH₂, NHalk and N(alk)₂ radicals, and alkyl, phenyl, CH₂-phenyl and heteroaryl radicals, such that, in the latter radicals, the alkyl, phenyl and heteroaryl radicals are themselves optionally substituted with one or more radicals chosen from halogen atoms and the radicals: hydroxyl, alkyl and alkoxy containing from 1 to 4 carbon atoms, NH₂, NHalk and N(alk)₂;

all the alkyl (alk) or alkoxy radicals above containing from 1 to 6 carbon atoms,

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above, in which:

Ra represents a hydrogen atom; a halogen atom; or an aryl or heteroaryl radical, these aryl and heteroaryl radicals being optionally substituted as indicated hereinafter;

Rb represents a hydrogen atom, a —CO-Rc radical or a —CO—NRcRd radical;

where Rc represents an alkyl radical or a cycloalkyl radical, both optionally substituted with one or more radicals chosen from hydroxyl, alkoxy, NR1R2, heterocycloalkyl, aryl and heteroaryl radicals, themselves optionally substituted as indicated hereinafter;

Rd represents a hydrogen atom or an alkyl radical;

all the alkyl, cycloalkyl, heterocycloalkyl, aryl and heteroaryl radicals defined above being optionally substituted with one or more radicals chosen from halogen atoms, and hydroxyl, alkoxy, —NR1R2, —COOH, —COOalk and —CONR1R2 radicals;

NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;

the cyclic radicals that R1 and R2 or R3 and R4, respectively, can form, with the nitrogen atom to which they are attached, being optionally substituted with one or more radicals, which may be identical or different, chosen from halogen atoms, hydroxyl and alkoxy radicals, and alkyl, phenyl and CH₂-phenyl radicals, in which the alkyl or phenyl radicals are themselves optionally substituted with one or more radicals, which may be identical or different, chosen from halogen atoms and alkyl, hydroxyl, alkoxy, NH₂, NHalk and N(alk)₂ radicals;

all the alkyl (alk) or alkoxy radicals above containing from 1 to 6 carbon atoms,

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above, in which:

Ra represents a hydrogen atom; a halogen atom; or a phenyl or pyrazolyl radical optionally substituted with one or more radicals chosen from halogen atoms, and the radicals: hydroxyl, alkoxy, —NR1R2, —COOH, —COOalk, —CONR1R2, alkyl and heterocycloalkyl, itself optionally substituted with one or more radicals chosen from halogen atoms, and alkyl, COOH, —COOalk and —CONR3R4 radicals;

Rb represents a hydrogen atom, a —CO-Rc radical or a —CO—NRcRd radical;

where Rc represents an alkyl or cycloalkyl radical, both optionally substituted with one or more radicals chosen from the radicals: hydroxyl, alkoxy, NR1R2 and phenyl, itself optionally substituted with one or more radicals chosen from halogen atoms, and hydroxyl, alkoxy, alkyl, NH₂, NHalk and N(alk)₂ radicals;

Rd represents a hydrogen atom or an alkyl radical;

NR1R2 is such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from the radicals: hydroxyl, alkoxy, NR3R4, or phenyl, itself optionally substituted with one or more radicals chosen from halogen atoms and hydroxyl, alkyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, this radical, including the possible NH that it contains being optionally substituted;

NR3R4 being such that: either R3 and R4, which may be identical or different, represent a hydrogen atom or an alkyl radical optionally substituted with one or more radicals, which may be identical or different, chosen from hydroxyl or alkoxy radicals; or R3 and R4 form, with the nitrogen atom to which they are attached, a cyclic radical containing from 4 to 7 ring members and optionally another heteroatom chosen from O, S, N and NH, this radical, including the possible NH that it contains, being optionally substituted;

The cyclic radicals that R1 and R2 or R3 and R4, respectively, can form, with the nitrogen atom to which they are attached, being optionally substituted with one or more radicals, which may be identical or different, as defined above;

all the alkyl (alk) or alkoxy radicals above containing from 1 to 4 carbon atoms,

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above, in which:

Ra represents a hydrogen atom; a halogen atom; or a phenyl or pyrazolyl radical optionally substituted with one or more radicals chosen from halogen atoms and the radicals: alkyl and heterocycloalkyl, itself optionally substituted with one or more radicals chosen from halogen atoms and alkyl and —COOalk radicals;

Rb represents a hydrogen atom, a —CO-Rc radical or a —CO—NRcRd radical;

where Rc represents an alkyl or cycloalkyl radical optionally substituted with one or more radicals chosen from hydroxyl, alkoxy and NR1R2 radicals;

Rd represents a hydrogen atom;

NR1R2 being such that: either R1 and R2, which may be identical or different, represent a hydrogen atom or an alkyl radical optionally substituted with one or more radicals, which may be identical or different, chosen from hydroxyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, optionally substituted with an alkyl, phenyl or —CH₂-phenyl radical, the latter radicals being themselves optionally substituted with one or more radicals, which may be identical or different, chosen from halogen atoms and alkyl, hydroxyl, alkoxy, NH₂, NHalk and N(alk)₂ radicals;

all the alkyl (alk) or alkoxy radicals above containing from 1 to 4 carbon atoms,

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; a halogen atom; or a phenyl radical which is optionally substituted as indicated hereinafter;

Rb represents a hydrogen atom, —CO-Rc radical or a —CO—NRcRd radical;

where Rc represents an alkyl or cycloalkyl radical, both optionally substituted with one or more radicals chosen from the radicals: hydroxyl, alkoxy, NR1R2 and phenyl, itself optionally substituted with one or more radicals chosen from halogen atoms, and hydroxyl, alkoxy, alkyl, NH₂, NHalk and N(alk)₂ radicals;

Rd represents a hydrogen atom or an alkyl radical;

NR1R2 is such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from the radicals: hydroxyl, alkoxy, NR3R4, or phenyl, themselves optionally substituted; or R1 and R2 form, with the nitrogen atom to which they are attached, a cyclic radical containing from 4 to 7 ring members and optionally another heteroatom chosen from O, S, N and NH, this radical, including the possible NH that it contains, being optionally substituted;

NR3R4 being such that: either R3 and R4, which may be identical or different, represent a hydrogen atom or an alkyl radical optionally substituted with one or more radicals, which may be identical or different, chosen from hydroxyl or alkoxy radicals; or R3 and R4 form, with the nitrogen atom to which they are attached, a cyclic radical containing from 4 to 7 ring members and optionally another heteroatom chosen from O, S, N and NH, this radical, including the possible NH that it contains, being optionally substituted;

the cyclic radicals that R1 and R2 or R3 and R4, respectively, can form, with the nitrogen atom to which they are attached, being optionally substituted with one or more radicals which may be identical or different, as defined above;

all the alkyl or alkoxy radicals above containing from 1 to 4 carbon atoms;

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom; a halogen atom; or a phenyl radical optionally substituted with a halogen atom;

Rb represents a hydrogen atom, a —CO-Rc radical or a —CO—NRcRd radical;

where Rc represents an alkyl or cycloalkyl radical optionally substituted with one or more radicals chosen from hydroxyl, alkoxy and NR1R2 radicals;

Rd represents a hydrogen atom;

NR1R2 being such that: either R1 and R2, which may be identical or different, represent a hydrogen atom or an alkyl radical optionally substituted with one or more radicals, which may be identical or different, chosen from hydroxyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, optionally substituted with an alkyl, phenyl or —CH₂-phenyl radical, the latter radicals being themselves optionally substituted with one or more radicals, which may be identical or different, chosen from halogen atoms and alkyl, hydroxyl, alkoxy, NH₂, NHalk and N(alk)₂ radicals;

all the alkyl (alk) or alkoxy radicals above containing from 1 to 4 carbon atoms;

said products of formula (I) being in all the possible racemic, enantiomeric and diastereoisomeric isomer forms, and also the addition salts with inorganic and organic acids or with inorganic and organic bases of said products of formula (I).

In the products of formula (I) and in the text hereinbelow:
the term “alkyl (or alk) radical” denotes linear and, where appropriate, branched methyl, ethyl, propyl, isopropyl, butyl, isobutyl, sec-butyl, tert-butyl, pentyl, isopentyl, hexyl, isohexyl and also heptyl, octyl, nonyl and decyl radicals and also the linear or branched positional isomers thereof: alkyl radicals containing from 1 to 6 carbon atoms and more particularly alkyl radicals containing from 1 to 4 carbon atoms of the above list are preferred;
the term “alkoxy radical” denotes linear and, where appropriate, branched methoxy, ethoxy, propoxy, isopropoxy, linear, secondary or tertiary butoxy, pentoxy or hexoxy radicals and also the linear or branched positional isomers thereof: alkoxy radicals containing from 1 to 4 carbon atoms of the above list are preferred;
the term “halogen atom” denotes chlorine, bromine, iodine or fluorine atoms, and preferably the chlorine, bromine or fluorine atom;
the term “cycloalkyl radical” denotes a saturated carbocyclic radical containing 3 to 10 carbon atoms and thus denotes in particular cyclopropyl, cyclobutyl, cyclopentyl and cyclohexyl radicals, and most particularly cyclopropyl, cyclopentyl and cyclohexyl radicals;
the term “heterocycloalkyl radical” thus denotes a monocyclic or bicyclic carbocyclic radical containing from 3 to 10 ring members, interrupted with one or more heteroatoms, which may be identical or different, chosen from oxygen, nitrogen or sulphur atoms: mention may, for example, be made of morpholinyl, thiomorpholinyl, homomorpholinyl, aziridyl, azetidyl, piperazinyl, piperidyl, homopiperazinyl, pyrrolidinyl, imidazolidinyl, pyrazolidinyl, tetrahydrofuryl, tetrahydrothienyl, tetrahydropyran, oxodihydropyridazinyl or else oxetanyl radicals, all these radicals being optionally substituted; mention may in particular be made of morpholinyl, thiomorpholinyl, homomorpholinyl, piperazinyl, piperidyl, homopiperazinyl or else pyrrolidinyl radicals;
the terms “aryl” and “heteroaryl” denote monocyclic or bicyclic, unsaturated or partially unsaturated, respectively carbocyclic and heterocyclic radicals containing at most 12 ring members, which may optionally contain a —C(O) ring member, the heterocyclic radicals containing one or more heteroatoms, which may be identical or different, chosen from O, N or S with N, where appropriate, optionally substituted;
the term “aryl radical” thus denotes monocyclic or bicyclic radicals containing 6 to 12 ring members, such as, for example, phenyl, naphthyl, biphenyl, indenyl, fluorenyl and anthracenyl radicals, more particularly phenyl and naphthyl radicals, and even more particularly the phenyl radical. It may be noted that a carbocyclic radical containing a —C(O) ring member is, for example, the tetralone radical;
the term “heteroaryl radical” thus denotes monocyclic or bicyclic radicals containing 5 to 12 ring members: monocyclic heteroaryl radicals, for instance the radicals: thienyl such as 2-thienyl and 3-thienyl, furyl such as 2-furyl or 3-furyl, pyrannyl, pyrrolyl, pyrrolinyl, pyrazolinyl, imidazolyl, pyrazolyl, pyridyl such as 2-pyridyl, 3-pyridyl and 4-pyridyl, pyrazinyl, pyrimidinyl, pyridazinyl, oxazolyl, thiazolyl, isothiazolyl, diazolyl, thiadiazolyl, thiatriazolyl, oxadiazolyl, isoxazolyl such as 3- or 4-isoxazolyl, furazanyl or tetrazolyl, which may be free or salified, all these radicals being optionally substituted, among which more particularly the radicals: thienyl such as 2-thienyl and 3-thienyl, furyl such as 2-furyl, pyrrolyl, pyrrolinyl, pyrazolinyl, imidazolyl, pyrazolyl, oxazolyl, isoxazolyl, pyridyl, pyridazinyl, these radicals being optionally substituted; bicyclic heteroaryl radicals, for instance the radicals: benzothienyl such as 3-benzothienyl, benzothiazolyl, quinolyl, isoquinolyl, dihydroquinolyl, quinolone, tetralone, adamentyl, benzofuryl, isobenzofuryl, dihydrobenzofuran, ethylenedioxyphenyl, thianthrenyl, benzopyrrolyl, benzimidazolyl, benzoxazolyl, thionaphthyl, indolyl, azaindolyl, indazolyl, purinyl, thienopyrazolyl, tetrahydroindazolyl, tetrahydrocyclopentapyrazolyl, dihydrofuropyrazolyl, tetrahydropyrrolopyrazolyl, oxotetrahydropyrrolo-pyrazolyl, tetrahydropyranopyrazolyl, tetrahydropyridinopyrazolyl or oxodihydropyridinopyrazolyl, all these radicals being optionally substituted.

As examples of heteroaryl or bicyclic radicals, mention may more particularly be made of pyrimidinyl, pyridyl, pyrrolyl, azaindolyl, indazolyl, pyrazolyl, benzothiazolyl or benzimidazolyl radicals, optionally substituted with one or more substituents, which may be identical or different, as indicated above.

The carboxyl radical(s) of the products of formula (I) may be salified or esterified with the various groups known to those skilled in the art, among which mention may, for example, be made of:
among the salification compounds, inorganic bases such as, for example, an equivalent of sodium, of potassium, of lithium, of calcium, of magnesium or of ammonium or organic bases such as, for example, methylamine, propylamine, trimethylamine, diethylamine, triethylamine, N,N-dimethyl-ethanolamine, tris(hydroxymethyl)aminomethane, ethanolamine, pyridine, picoline, dicyclohexylamine, morpholine, benzylamine, procaine, lysine, arginine, histidine or N-methylglucamine,
among the esterification compounds, alkyl radicals for forming alkoxycarbonyl groups, such as, for example, methoxycarbonyl, ethoxycarbonyl, tert-butoxycarbonyl or benzyloxycarbonyl, these alkyl radicals possibly being substituted with radicals chosen, for example, from halogen atoms, and hydroxyl, alkoxy, acyl, acyloxy, alkylthio, amino or aryl radicals, such as for instance in chloromethyl, hydroxypropyl, methoxymethyl, propionyloxymethyl, methylthiomethyl, dimethylaminoethyl, benzyl or phenethyl groups.

The addition salts with inorganic or organic acids of the products of formula (I) may, for example, be the salts formed with hydrochloric acid, hydrobromic acid, hydroiodic acid, nitric acid, sulphuric acid, phosphoric acid, propionic acid, acetic acid, trifluoroacetic acid, formic acid, benzoic acid, maleic acid, fumaric acid, succinic acid, tartaric acid, citric acid, oxalic acid, glyoxylic acid, aspartic acid, ascorbic acid, alkylmonosulphonic acids such as, for example, methanesulphonic acid, ethanesulphonic acid or propanesulphonic acid, alkyldisulphonic acids such as, for example, methanedisulphonic acid or alpha,beta-ethanedisulphonic acid, arylmonosulphonic acids such as benzenesulphonic acid and aryldisulphonic acids.

It may be recalled that stereoisomerism can be defined in its broad sense as the isomerism of compounds having the same structural formulae, but the various groups of which are arranged differently in space, such as in particular in monosubstituted cyclohexanes, the substituent of which can be in the axial or equatorial position, and the various possible rotational conformations of ethane derivatives. However, another type of stereoisomerism exists, due to the different spatial arrangements of substituents attached either on double bonds or on rings, which is commonly known as geometrical isomerism or cis-trans isomerism. The term stereoisomers is used in the present application in its broadest sense and therefore relates to all the compounds indicated above.

When NR1R2 or NR3R4 forms a ring as defined above, such an aminated ring may be chosen, in particular, from pyrrolidinyl, pyrazolidinyl, pyrazolinyl, piperidyl, azepinyl, morpholinyl, homomorpholinyl, piperazinyl or homopiperazinyl radicals, these radicals being themselves optionally substituted as indicated above or hereinafter: for example, with one or more radicals, which may be identical or different, chosen from halogen atoms and alkyl, hydroxyl, alkoxy, phenyl and CH₂-phenyl radicals, the alkyl or phenyl radicals being themselves optionally substituted with one or more radicals, which may be identical or different, chosen from halogen atoms and alkyl, hydroxyl, alkoxy, NH₂, NHalk and N(alk)₂ radicals.

The NR1R2 or NR3R4 ring may more particularly be chosen from pyrrolidinyl radicals or morpholino radicals, optionally substituted with one or two alkyl radicals or piperazinyl radicals, optionally substituted on the second nitrogen atom with an alkyl, phenyl, or CH₂-phenyl radical, themselves optionally substituted with one or more radicals, which may be identical or different, chosen from halogen atoms and alkyl, hydroxyl and alkoxy radicals.

A subject of the present invention is the products of formula (I) as defined above, in which:

Ra represents a hydrogen atom or a phenyl or pyrazolyl radical optionally substituted with one or more radicals chosen from halogen atoms and the radicals: alkyl and piperidyl, itself optionally substituted with —COOalk;

Rb represents a hydrogen atom, a —CO-Rc radical or a —CO—NRcRd radical;

where Rc represents a cyclopropyl radical or an alkyl radical optionally substituted with an alkoxy or NR1R2 radical;

Rd represents a hydrogen atom;

NR1R2 being such that: either R1 and R2, which may be identical or different, represent a hydrogen atom or an alkyl radical; or R1 and R2 form, with the nitrogen atom to which they are attached, a morpholinyl radical;

the alkyl or alkoxy radicals above containing from 1 to 4 carbon atoms;

said products of formula (I) being in all the possible racemic, enantiomeric and diastereoisomeric isomer forms, and also the addition salts with inorganic and organic acids or with inorganic and organic bases of said products of formula (I).

A subject of the present invention is the products of formula (I) as defined above or hereinafter, in which:

Ra represents a hydrogen atom or a phenyl radical optionally substituted with a halogen atom;

Rb represents a hydrogen atom, a —CO-Rc radical or a —CO—NRcRd radical;

where Rc represents a cyclopropyl radical or an alkyl radical optionally substituted with an alkoxy or NR1R2 radical;

Rd represents a hydrogen atom; NR1R2 being such that: either R1 and R2, which may be identical or different, represent a hydrogen atom or an alkyl radical; or R1 and R2 form, with the nitrogen atom to which they are attached, a morpholinyl radical;

the alkyl or alkoxy radicals above containing from 1 to 4 carbon atoms;

said products of formula (I) being in all the possible racemic, enantiomeric and diastereoisomeric isomer forms, and also the addition salts with inorganic and organic acids or with inorganic and organic bases of said products of formula (I).

A subject of the present invention is most particularly the products of formula (I) as defined above, corresponding to the following formulae:
N-{[6-(imidazo[1,2-a]pyridin-3-yl)sulphanyl]-1,3-benzothiazol-2-yl}cyclopropanecarboxamide
6-{[6-(4-fluorophenyl)imidazo[1,2-a]pyridin-3-yl]sulphanyl}-1,3-benzothiazol-2-amine
N-(6-{[6-(4-fluorophenyl)imidazo[1,2-a]pyridin-3-yl]sulphanyl}-1,3-benzothiazol-2-yl)cyclopropanecarboxamide
N-(6-{[6-(4-fluorophenyl)imidazo[1,2-a]pyridin-3-yl]sulphanyl}-1,3-benzothiazol-2-yl)acetamide
1-(6-{[6-(4-fluorophenyl)imidazo[1,2-a]pyridin-3-yl]sulphanyl}-1,3-benzothiazol-2-yl)-3-(2-methoxyethyl)urea
1-(6-{[6-(4-fluorophenyl)imidazo[1,2-a]pyridin-3-yl]sulphanyl}-1,3-benzothiazol-2-yl)-3-[2-(morpholin-4-yl)ethyl]urea
N-(6-{[6-(1-methyl-1H-pyrazol-4-yl)imidazo[1,2-a]pyridin-3-yl]sulphanyl}-1,3-benzothiazol-2-yl)cyclopropanecarboxamide
N-(6-{[6-(1H-pyrazol-4-yl)imidazo[1,2-a]pyridin-3-yl]sulphanyl}-1,3-benzothiazol-2-yl)cyclopropanecarboxamide
N-(6-{[6-((3-fluorophenyl)imidazo[1,2-a]pyridin-3-yl]sulphanyl}-1,3-benzothiazol-2-yl)cyclopropanecarboxamide
N-(6-{[6-((3-fluoro-4-methyl)phenyl)imidazo[1,2-a]pyridin-3-yl]sulphanyl}-1,3-benzothiazol-2-yl)cyclopropanecarboxamide
tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}piperidine-1-carboxylate
N-[6-({6-[1-(piperidin-4-yl)-1H-pyrazol-4-yl]imidazo[1,2-a]pyridin-3-yl}sulphanyl)-1,3-benzothiazol-2-yl]cyclopropanecarboxamide
   and also the addition salts with inorganic and organic acids or with inorganic and organic bases of said products of formula (I).

Another subject of the present invention is any process for preparing the products of formula (I) as defined above.

The products according to the invention can be prepared on the basis of conventional organic chemistry methods.

### Preparation of Compounds of Formula (I)

Schemes 1, 2 and 3 below illustrate the methods used to prepare the products of formula (I). In this respect, they cannot constitute a limitation of the scope of the invention, with regard to the methods of preparation of the compounds claimed.

The products of formula (I) as defined above according to the present invention can thus in particular be prepared according to the process described in schemes 1, 2 and 3 below.

A subject of the present invention is thus also the process for preparing products of formula (I) according to scheme 1 as defined hereinafter.

A subject of the present invention is thus also the process for preparing products of formula (I) according to scheme 2 as defined hereinafter.

A subject of the present invention is thus also the process for preparing products of formula (I) according to scheme 3 as defined hereinafter.

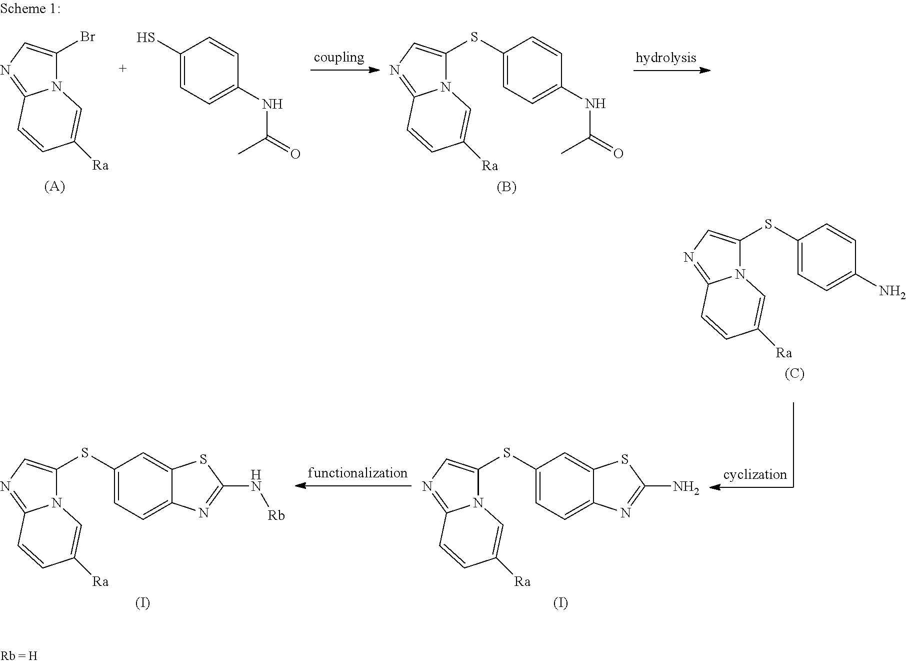

In scheme 1 above, the substituents Ra and Rb have the meanings indicated above.

The compounds (I) for which Ra and Rb have the same meanings can be obtained from the compounds (I) for which Rb=H.

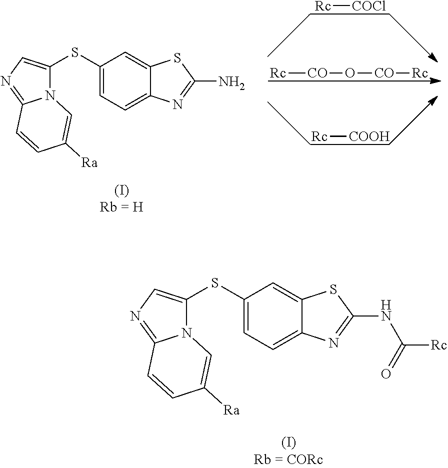

More particularly, the compounds (I) for which Rb=CORc (with Rc as defined above) can be obtained, for example:
by reacting an acid chloride of formula Rc-COCl in the presence, for example, of a solvent such as pyridine at a temperature in the region of 20° C.,
by reacting an acid anhydride of formula Rc-CO—O—CO-Rc, in the presence, for example, of a solvent such as pyridine at a temperature in the region of 20° C.,
by reacting with a carboxylic acid of formula Rc-COOH under the conditions, for example, described by D. DesMarteau et al. (Chem. Let, 2000, 9, 1052) in the presence of 1-hydroxybenzotriazole and of 1-(3-dimethylaminopropyl)-3-ethylcarbodiimide and in the presence of a base such as triethylamine, at a temperature between 20° C. and the reflux temperature of the solvent.

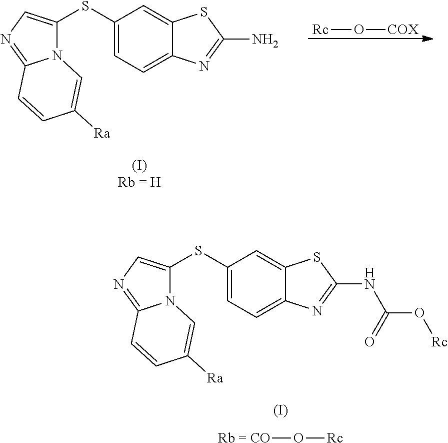

More particularly, the compounds (I) for which Rb=CO—O-Rc (with Rc as defined above) can be obtained, for example, by reaction with a chlorocarbonate Rc-O—COX (X=Cl) on the compounds (I) for which Rb=H, in a solvent such as tetrahydrofuran, in the presence of a base such as sodium hydrogen carbonate, or in pyridine, at a temperature in the region of 20° C.

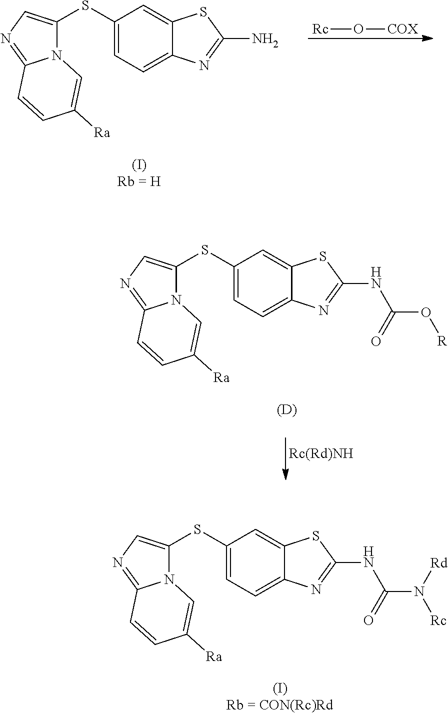

More particularly, the compounds (I) for which Rb=CON(Rc)Rd (with Rc and Rd as defined above) can be obtained, for example, by reacting the carbamates (D) where R=phenyl, with amines Rc (Rd)NH (with Rc and Rd as defined above), in the presence of an aprotic solvent such as tetrahydrofuran, at a temperature in the region of 20° C.

The carbamates (D) can be obtained, for example, by reaction with a chlorocarbonate R—O—COX (X=Cl) on the compounds (I) for which Rb=H, in a solvent such as tetrahydrofuran, in the presence of a base such as sodium hydrogen carbonate, or in pyridine, at a temperature in the region of 20° C.

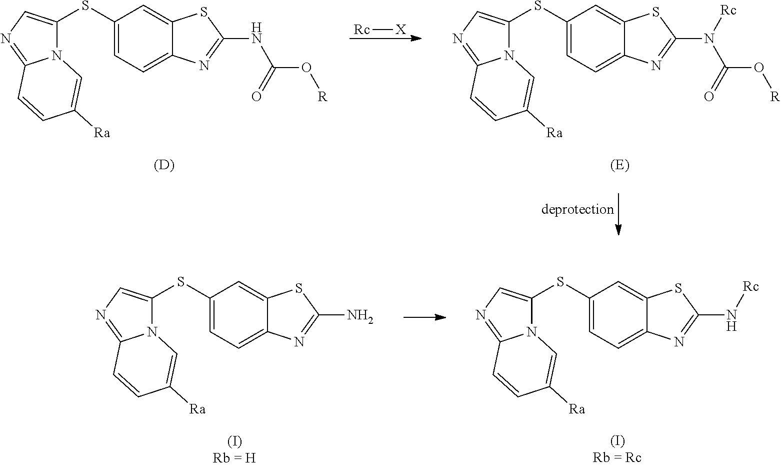

More particularly, the compounds (I) for which Rb=Rc (with Rc as defined above) can be obtained, for example:
by deprotection of the carbamates (E) with R=t-butyl according to a customary method for those skilled in the art, for example with trifluoroacetic acid, in a solvent such as dichloromethane at a temperature in the region of 20° C.
from the compounds (I) for which Rb=H, by application of the methods described in patent EP 0408437 or by R. A Glennon et al. (Journal of Medicinal Chemistry, 1981, 24, 766-769).

The carbamates (E) can be obtained, for example, by reacting the carbamates (D) where R=t-butyl, with halides Rc-X (with Rc as defined above), in the presence of a solvent such as N,N-dimethylformamide, in the presence of a base such as sodium hydride, at a temperature of between 20° C. and 90° C.

The compounds (I) for which Rb=H can be obtained by cyclization of the compounds (C) according to a customary method for those skilled in the art, for example by application of the methods described by H. Masaichi et al. (Journal of Medicinal Chemistry, 2007, 50(18), 4453-4470), by reacting potassium thiocyanate and bromine in the presence of an acid such as acetic acid, at a temperature of between 20° C. and the reflux temperature of the solvent.

The compounds (C) can be obtained by hydrolysis of the acetamide function of the compounds (B) according to a customary method for those skilled in the art, for example using an acid such as hydrochloric acid, in a solvent such as ethanol, at a temperature of between 20° C. and the reflux of the solvent.

The compounds (B) can be obtained by coupling the compounds (A), with Ra as defined above, with N-(4-sulphanylphenyl)acetamide (commercial product), under the conditions described, for example, by R. Varala et al. (Chemistry Letters, 2004, 33(12), 1614-1615), and by M. Winn et al. (Journal of Medicinal Chemistry, 2001, 44, 4393-4403), in the presence of a base such as, for example, potassium carbonate, in a solvent such as dimethyl sulphoxide, at a temperature of between 20° C. and the reflux temperature of the solvent. Such reactions can also be carried out under microwave irradiation.

The compounds (B) can also be obtained by coupling the compounds (A) as described above with other 4-aminothiophenol derivatives such as (4-NHR)Ph-SH derivatives where the amine function is free ((4-NH₂)Ph-SH, commercial product) or protected with a t-butyloxycarbonyl group, for example ((4-NHCO₂tBu)Ph-SH, known product).

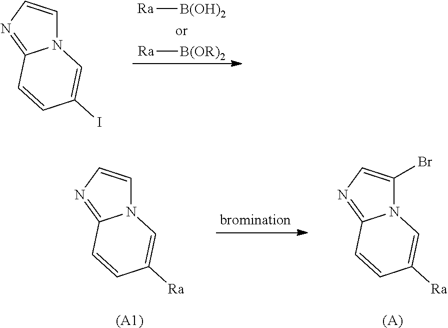

The compounds (A) are either commercially available (Ra=H), or prepared by bromination of the compounds (A1), according to a customary method for those skilled in the art, for example according to the conditions described by E. S. Hand et al. (Journal of Organic Chemistry, 1980, 45, 3738-3745), or using bromine in a solvent such as ethanol, at a temperature of between 20° C. and the reflux of the solvent.

The compounds (A1) are either commercially available (Ra=H), or can be obtained from 6-iodoimidazo[1,2-a]pyridine (known compound, the preparation of which is described by C. Enguehard et al., Helvetica Chimica Acta (2001), 84, 3610-3614) by means of a coupling reaction by application of the methods described by C. Enguehard et al. (Helvetica Chimica Acta (2001), 84, 3610-3614), for example:
using the boronic acids of formula Ra-B(OH)₂ in the presence of sodium hydrogen carbonate and of tetrakis(triphenylphosphine)palladium in a solvent such as dimethyl sulphoxide or dioxane, at a temperature in the region of 80° C.,
using the boronic esters Ra-B(OR)₂ in the presence of dichlorobis(triphenylphosphine)palladium in a solvent such as, for example, 1,2-dimethoxyethane, in the presence of a base such as 1N sodium hydroxide, at a temperature in the region of 80° C.

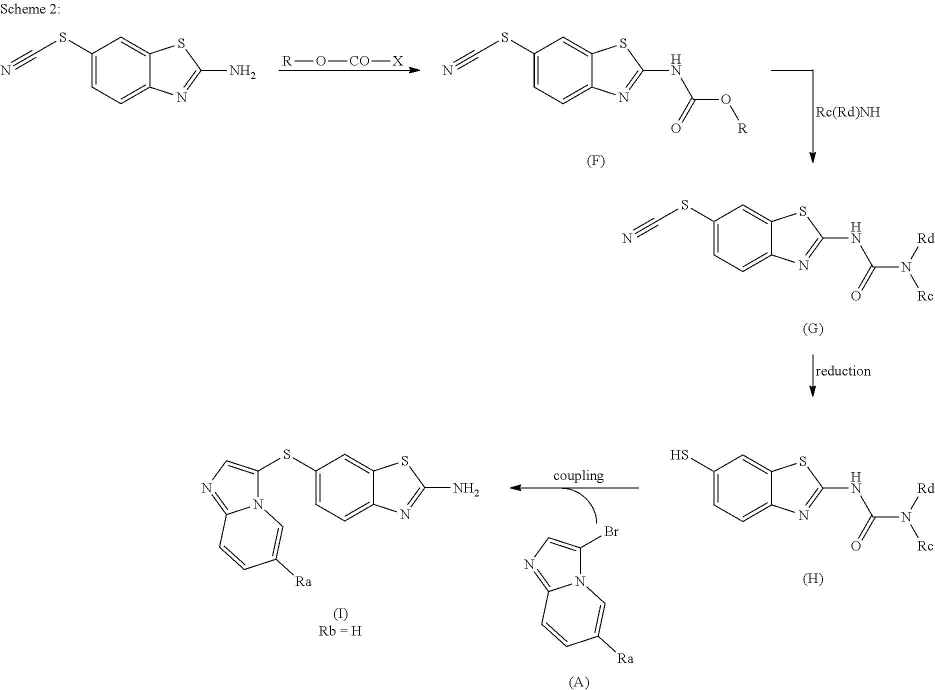

In scheme 2 above, the substituents Ra, Rc and Rd having the meanings indicated above.

The compounds (I) for which Ra has the same meanings as above and for which Rb=H can be obtained by means of a coupling reaction of the compounds (A) with Ra as defined above, with the Compounds (H) with Rc and Rd as defined above, as described above for the preparation of the compounds (B).

The compounds (H) for which Rc and Rd have the same meanings indicated above can be obtained, for example, by reduction of the compounds (G) with DL-dithiotreitol, in the presence of sodium hydrogen carbonate or potassium dihydrogen phosphate, in a solvent such as ethanol and at a temperature of between 20° C. and the reflux of the solvent.

The compounds (G) for which Rc and Rd have the same meanings indicated above can be obtained, for example, from the compounds (F), as described above for the preparation of the compounds (I) with Rb=CO—N(Rc)Rd.

The compounds (F) can be obtained using 2-amino-1,3-benzothiazol-6-yl thiocyanate (commercial product) as described above for the preparation of the compounds (D).

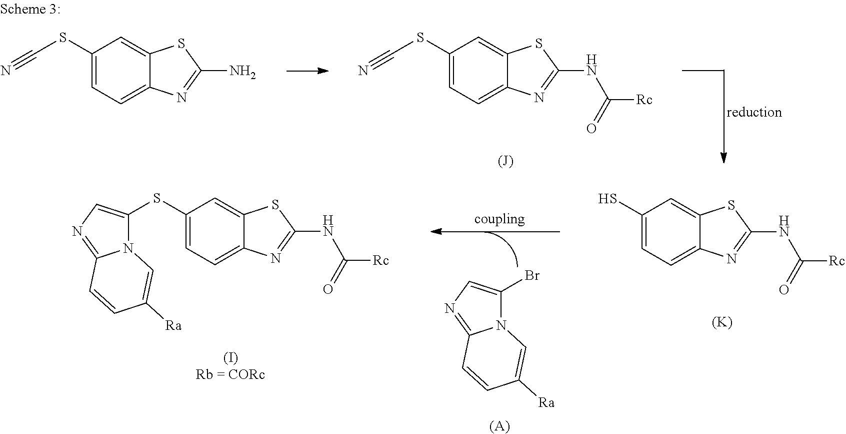

In scheme 3 above, the substituents Ra and Rc have the meanings indicated above.

The compounds (I) for which Ra has the same meanings as above and for which Rb=CORc, can be obtained by means of a coupling reaction of the compounds (A) with Ra as defined above, with the compounds (K) with Rc as defined above, as described for the preparation of the compounds (B).

The compounds (K) for which Rc has the same meanings indicated above can be obtained, for example, by reduction of the compounds (J) with DL-dithiotreitol, in the presence of sodium hydrogen carbonate or potassium dihydrogen phosphate, in a solvent such as ethanol, and at a temperature of between 20° C. and the reflux of the solvent.

The compounds (J) for which Rc has the same meanings indicated above can be obtained from 2-amino-1,3-benzothiazol-6-yl thiocyanate (commercial product), as described above for the preparation of the compounds (I), with Rb=CORc, from the compounds (I) with Rb=H.

Among the starting products of formulae (A), (A1), (A2), (F), (G), (J) and (K), some are known and can be obtained either commercially or according to the usual methods known to those skilled in the art, for example from commercial products.

It is understood, for those skilled in the art, that, in order to carry out the processes according to the invention described above, it may be necessary to introduce protective groups for amino, carboxyl and alcohol functions in order to avoid side reactions.

The following nonexhaustive list of examples of protection of reactive functions may be mentioned:
hydroxyl groups may be protected, for example, with alkyl radicals such as tert-butyl, trimethysilyl, tert-butyldimethylsilyl, methoxymethyl, tetrahydropyranyl, benzyl or acetyl,

amino groups may be protected, for example, with acetyl, trityl, benzyl, tert-butoxycarbonyl (BOC), benzyloxycarbonyl or phthalimido radicals or other radicals known in peptide chemistry.

Acid functions may be protected, for example, in the form of esters formed with readily cleavable esters such as benzyl or tert-butyl esters or esters known in peptide chemistry.

A list of various protective groups that may be used will be found in the textbooks known to those skilled in the art and, for example, in patent BF 2 499 995.

It may be noted that it is possible, if desired and if necessary, to subject intermediate products or products of formula (I) thus obtained by the processes indicated above, in order to obtain other intermediates or other products of formula (I), to one or more conversion reactions known to those skilled in the art, for instance:

a) a reaction for esterification of an acid function,

b) a reaction for saponification of an ester function to give an acid function,

c) a reaction for reducing a free or esterified carboxyl function to give an alcohol function,

d) a reaction for conversion of an alkoxy function to give a hydroxyl function, or alternatively of a hydroxyl function to give an alkoxy function,

e) a reaction for removal of the protective groups that may be borne by the protected reactive functions,

f) a reaction for salification with an inorganic or organic acid or with a base so as to obtain the corresponding salt,

g) a reaction for resolution of the racemic forms to give resolved products, said products of formula (I) thus obtained being in all the possible racemic, enantiomeric and diastereoisomeric isomer forms.

The reactions a) to g) can be carried out under the usual conditions known to those skilled in the art, for instance those indicated hereinafter.

a) The products described above may, if desired, undergo, on the possible carboxyl functions, esterification reactions that may be performed, according to the usual methods known to those skilled in the art.

b) The possible conversions of ester functions to give acid functions of the products described above may, if desired, be performed under the usual conditions known to those skilled in the art, in particular by acid or alkaline hydrolysis, for example with sodium hydroxide or potassium hydroxide in an alcoholic medium, for instance in methanol, or alternatively with hydrochloric acid or sulphuric acid.

The saponification reaction may be carried out according to the usual methods known to those skilled in the art, for instance in a solvent such as methanol or ethanol, dioxane or dimethoxyethane, in the presence of sodium hydroxide or potassium hydroxide.

c) The possible free or esterified carboxyl functions of the products described above may be reduced, if desired, to give alcohol functions via the methods known to those skilled in the art; the possible esterified carboxyl functions may be reduced, if desired, to give alcohol functions by the methods known to those skilled in the art, and in particular with lithium aluminium hydride in a solvent such as, for example, tetrahydrofurane, or else dioxane or ethyl ether.

The possible free carboxyl functions of the products described above may be reduced, if desired, to give alcohol functions, in particular with boron hydride.

d) The possible alkoxy functions, such as in particular methoxy, of the products described above may be converted, if desired, into hydroxyl functions under the usual conditions known to those skilled in the art, for example with boron tribromide in a solvent such as, for example, methylene chloride, with pyridine hydrochloride or hydrobromide, or alternatively with hydrobromic acid or hydrochloric acid in water or trifluoroacetic acid at reflux.

e) The removal of protective groups, for instance those indicated above, may be carried out under the usual conditions known to those skilled in the art, in particular via an acid hydrolysis performed with an acid such as hydrochloric acid, benzenesulphonic or para-toluenesulphonic acid, formic acid or trifluoroacetic acid, or alternatively via catalytic hydrogenation.

The phthalimido group may be removed with hydrazine.

f) The products described above may, if desired, undergo salification reactions, for example with an inorganic or organic acid or with an inorganic or organic base according to the usual methods known to those skilled in the art: such a salification reaction may be carried out, for example, in the presence of hydrochloric acid, or alternatively of tartaric acid, citric acid or methanesulphonic acid, in an alcohol such as, for example, ethanol or methanol.

g) The possible optically active forms of the products described above may be prepared by resolution of the racemic mixtures according to the usual methods known to those skilled in the art.

The products of formula (I) as defined above and also the addition salts thereof with acids exhibit advantageous pharmacological properties, in particular owing to their kinase-inhibiting properties as indicated above.

The products of the present invention can in particular be used for treating tumours.

The products of the invention may thus also increase the therapeutic effects of commonly used antitumour agents.

These properties justify their therapeutic use, and a subject of the invention is in particular, as medicaments, the products of formula (I) as defined above, said products of formula (I) being in all the possible racemic, enantiomeric and diastereoisomeric isomer forms, and also the addition salts with pharmaceutically acceptable inorganic and organic acids or with pharmaceutically acceptable inorganic and organic bases of said products of formula (I).

A subject of the invention is most particularly, as medicaments, the products corresponding to the following formulae:
N-{[6-(imidazo[1,2-a]pyridin-3-yl)sulphanyl]-3-benzothiazol-2-yl}cyclopropanecarboxamide
6-{[6-(4-fluorophenyl)imidazo[1,2-a]pyridin-3-yl]sulphanyl}-1,3-benzothiazol-2-amine
N-(6-{[6-(4-fluorophenyl)imidazo[1,2-a]pyridin-3-yl]sulphanyl}-1,3-benzothiazol-2-yl)cyclopropanecarboxamide
N-(6-{[6-(4-fluorophenyl)imidazo[1,2-a]pyridin-3-yl]sulphanyl}-1,3-benzothiazol-2-yl)acetamide
1-(6-{[6-(4-fluorophenyl)imidazo[1,2-a]pyridin-3-yl]sulphanyl}-1,3-benzothiazol-2-yl)-3-(2-methoxyethyl)urea
1-(6-{[6-(4-fluorophenyl)imidazo[1,2-a]pyridin-3-yl]sulphanyl}-1,3-benzothiazol-2-yl)-3-[2-(morpholin-4-yl)ethyl]urea
N-(6-{[6-(1-methyl-1H-pyrazol-4-yl)imidazo[1,2-a]pyridin-3-yl]sulphanyl}-1,3-benzothiazol-2-yl)cyclopropanecarboxamide
N-(6-{[6-(1H-pyrazol-4-yl)imidazo[1,2-a]pyridin-3-yl]sulphanyl}-1,3-benzothiazol-2-yl)cyclopropanecarboxamide
N-(6-{[6-(3-fluorophenyl)imidazo[1,2-a]pyridin-3-yl]sulphanyl}-1,3-benzothiazol-2-yl)cyclopropanecarboxamide
N-(6-{[6-((3-fluoro-4-methyl)phenyl)imidazo[1,2-a]pyridin-3-yl]sulphanyl}-1,3-benzothiazol-2-yl)cyclopropanecarboxamide
tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}piperidine-1-carboxylate
N-[6-({6-[1-(piperidin-4-yl)-1H-pyrazol-4-yl]imidazo[1,2-a]pyridin-3-yl}sulphanyl)-1,3-benzothiazol-2-yl]cyclopropanecarboxamide
   and also the addition salts with pharmaceutically acceptable inorganic and organic acids or with pharmaceutically acceptable inorganic and organic bases of said products of formula (I).

The invention also relates to pharmaceutical compositions containing, as active ingredient, at least one of the products of formula (I) as defined above or a pharmaceutically acceptable salt of this product or a prodrug of this product and, where appropriate, a pharmaceutically acceptable carrier.

The invention thus covers the pharmaceutical compositions containing, as active ingredient, at least one of the medicaments as defined above.

Such pharmaceutical compositions of the present invention may also, where appropriate, contain active ingredients of other antimitotic medicaments, such as, in particular, those based on taxol, cisplatin, DNA intercalating agents, and the like.

These pharmaceutical compositions may be administered orally, parenterally or locally by topical application to the skin and the mucous membranes or by intravenous or intramuscular injection.

These compositions may be solid or liquid and may be in any of the pharmaceutical forms commonly used in human medicine, for instance simple or sugar-coated tablets, pills, lozenges, gel capsules, drops, granules, injectable preparations, ointments, creams or gels; they are prepared according to the usual methods. The active ingredient may, therein, be incorporated into excipients normally used in these pharmaceutical compositions, such as talc, gum arabic, lactose, starch, magnesium stearate, cocoa butter, aqueous or nonaqueous carriers, fatty substances of animal or plant origin, paraffin derivatives, glycols, various wetting agents, dispersants or emulsifiers, and preservatives.

The usual dosage, which is variable depending on the product used, the individual treated and the condition in question, may, for example, be from 0.05 to 5 g per day in adults, or preferably from 0.1 to 2 g per day.

A subject of the present invention is also the use of the products of formula (I) as defined above or of pharmaceutically acceptable salts of these products, for the preparation of a medicament for use in inhibiting the activity of a protein kinase.

A subject of the present invention is also the use of products of formula (I) as defined above, for the preparation of a medicament for use in the treatment or prevention of a disease characterized by dysregulation of the activity of a protein kinase.

Such a medicament may in particular be for use in the treatment or prevention of a disease in a mammal.

A subject of the present invention is also the use defined above, in which the protein kinase is a protein tyrosine kinase.

A subject of the present invention is also the use defined above, in which the protein tyrosine kinase is MET or mutant forms thereof.

A subject of the present invention is also the use defined above, in which the protein kinase is in a cell culture.

A subject of the present invention is also the use defined above, in which the protein kinase is in a mammal.

A subject of the present invention is in particular the use of a product of formula (I) as defined above, for the preparation of a medicament for use in the prevention or treatment of diseases associated with an uncontrolled proliferation.

A subject of the present invention is in particular the use of a product of formula (I) as defined above, for the preparation of a medicament for use in the treatment or prevention of a disease chosen from the following group: blood vessel proliferation disorders, fibrotic disorders, ‘mesangial’ cell proliferation disorders, metabolic disorders, allergies, asthma, thrombosis, nervous system diseases, retinopathy, psoriasis, rheumatoid arthritis, diabetes, muscle degeneration and cancers.

A subject of the present invention is thus most particularly the use of a product of formula (I) as defined above, for the preparation of a medicament for use in the treatment or prevention of diseases in oncology, and in particular for use in the treatment of cancers.

Among these cancers, the treatment of solid or liquid tumours and the treatment of cancers that are resistant to cytotoxic agents are of interest.

The cited products of the present invention may in particular be used for the treatment of primary tumours and/or metastases, in particular gastric, hepatic, renal, ovarian, colon, prostate and lung (NSCLC and SCLC) cancers, glioblastomas, thyroid, bladder or breast cancers, in melanomas, in lymphoid or myeloid haematopoietic tumours, in sarcomas, in brain, larynx or lymphatic system cancers, bone cancers and pancreatic cancers.

A subject of the present invention is also the use of the products of formula (I) as defined above, for the preparation of medicaments for use in cancer chemotherapy.

Such medicaments for use in cancer chemotherapy may be used alone or in combination.

The products of the present application may in particular be administered alone or in combination with chemotherapy or radiotherapy or alternatively in combination, for example, with other therapeutic agents.

Such therapeutic agents may be commonly used antitumour agents.

As kinase inhibitors, mention may be made of butyrolactone, flavopiridol and 2-(2-hydroxyethylamino)-6-benzylamino-9-methylpurine, also known as olomoucine.

A subject of the present invention is also, as novel industrial products, the synthesis intermediates of formulae (A), (B), (C), (D), (E), (F), (G), (H), (J) and (K) as defined above and recalled hereinafter:

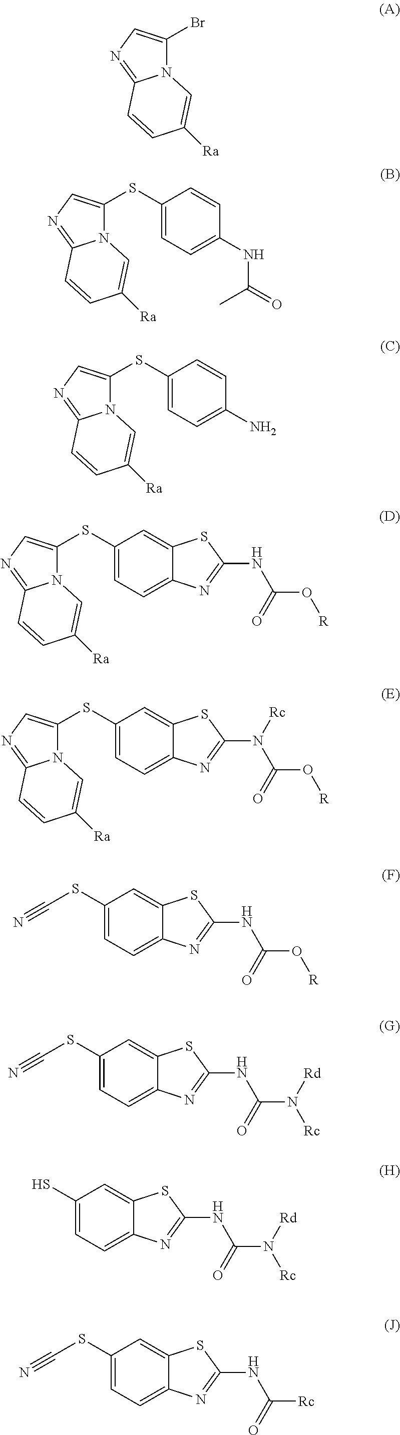

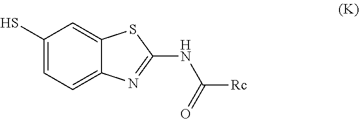

as defined above, in which Ra, Rb, Rc and Rd have the definitions indicated above, and R represents a t-butyl or phenyl radical.

The following examples, which are products of formula (I), illustrate the invention without, however, limiting it.

### EXPERIMENTAL SECTION

The nomenclature of the compounds of the present invention was carried out with the ACDLABS software, version 10.0.

The microwave oven used is a Biotage, Initiator™ 2.0 microwave device, 400 W max, 2450 MHz.

The ¹H NMR spectra at 400 MHz and the ¹H NMR spectra at 500 MHz were acquired on a Bruker Avance DRX-400 or Bruker Avance DPX-500 spectrometer with chemical shifts (δ in ppm) in the solvent d₆-dimethyl sulphoxide (DMSO-d₆) referenced at 2.5 ppm at a temperature of 303K.

The mass spectra (MS) were obtained either by method A or by method B:

### Method A:

Waters HPLC-SQD machine; ionization: positive and/or negative mode electrospray (ES+/−); chromatographic conditions: column: Acquity BEH C₁₈ 1.7 μm-2.1×50 mm; solvents: A: H₂O (0.1% formic acid) B: CH₃CN (0.1% formic acid); column temperature: 50° C.; flow rate: 1 ml/min; gradient (2 min): from 5 to 50% of B in 0.8 min; 1.2 min: 100% of B; 1.85 min: 100% of B; 1.95: 5% of B; retention time=Tr (min).

### Method B:

Waters ZQ machine; ionization: positive and/or negative mode electrospray (ES+/−); chromatographic conditions: column: XBridge C₁₈ 2.5 μm-3×50 mm; solvents: A: H₂O (0.1% formic acid) B: CH₃CN (0.1% formic acid); column temperature: 70° C.; flow rate: 0.9 ml/min; gradient (7 min): from 5 to 100% of B in 5.3 min; 5.5 min: 100% of B; 6.3 min: 5% of B; retention time=Tr (min).

### Example 1

### N-{[6-(imidazo[1,2-a]pyridin-3-yl)sulphanyl]-1,3-benzothiazol-2-yl}cyclopropanecarboxamide

### Example 1a

### N-{[6-(imidazo[1,2-a]pyridin-3-yl)sulphanyl]-1,3-benzothiazol-2-yl}cyclopropanecarboxamide

The compound can be prepared in the following way:

330 μl of cyclopropanecarbonyl chloride are added, dropwise, to a solution of 85 mg of 6-[imidazo[1,2-a]pyridin-3-yl)sulphanyl]-1,3-benzothiazol-2-amine and of 2 ml of pyridine. The reaction medium is stirred at a temperature in the region of 20° C. for 16 hours and then poured into 60 ml of water. The precipitate formed is filtered off, washed with 20 ml of water and 20 ml of a saturated aqueous solution of sodium hydrogen carbonate, spin-filter-dried, and dried. The solid isolated is taken up with 3 ml of isopropanol and brought to reflux. After a return to a temperature in the region of 20° C., the solid is filtered off, washed twice with 1 ml of isopropanol and twice with 3 ml of diethyl ether, spin-filter-dried, and then dried. 55 mg of N-{[6-(imidazo[1,2-a]pyridin-3-yl)sulphanyl]-1,3-benzothiazol-2-yl}cyclopropanecarboxamide are thus obtained in the form of a beige solid.

Melting point>260° C. (Köfler bench).

MS: method A; [M+H]⁺: m/z=367; [M−H]⁻: m/z=365; Tr=0.59 min.

¹H NMR (500 MHz, DMSO-d₆) δ ppm 0.88-1.11 (m, 4H) 1.96 (m, 1H) 7.06 (m, 1H) 7.15 (dd, J=8.5, 1.7 Hz, 1H) 7.43 (m, 1H) 7.61 (d, J=8.5 Hz, 1H) 7.73 (d, J=8.5 Hz, 1H) 7.83 (broad s, 1H) 8.08 (s, 1H) 8.42 (d, J=7.1 Hz, 1H) 12.59 (broad m, 1H).

### Example 1b

### 6-[imidazo[1,2-a]pyridin-3-yl)sulphanyl]-1,3-benzothiazol-2-amine

The compound can be prepared in the following way:

104 mg of 3-bromoimidazo[1,2-a]pyridine (commercial product), 171 mg of 1-[2-(morpholin-4-yl)ethyl]-3-(6-sulphanyl-1,3-benzothiazol-2-yl)urea, 140 mg of potassium carbonate and 2 ml of dimethyl sulphoxide are charged to a sealed glass tube. The medium is microwave-heated at 190° C. for 10 minutes. After a return to a temperature in the region of 20° C., the medium is poured into 50 ml of water and ice, and extracted with 3 times 25 ml of dichloromethane, and the combined organic, extracts are dried over magnesium sulphate, filtered, and concentrated to dryness under reduced pressure. The evaporation residue is chromatographed, under an argon pressure, on silica gel (eluent: 9/1 dichloromethane/methanol) and makes it possible to isolate a solid which is triturated from 2 ml of diisopropyl ether, filtered, washed twice with 2 ml of diisopropyl ether and dried. 19 mg of 6-[imidazo[1,2-a]pyridin-3-yl)sulphanyl]-1,3-benzothiazol-2-amine are thus obtained in the form of a cream solid.

Melting point=226° C. (Köfler bench).

MS: method A; [M+H]⁺: m/z=299; [M+2H]²⁺: m/z=150 (base peak); [M+CH3CN+2H]²⁺: m/z=170; Tr=0.37 min.

¹H NMR (400 MHz, DMSO-d₅) δ ppm 7.02-7.10 (m, 2H) 7.22 (d, J=8.3 Hz, 1H) 7.37-7.44 (m, 1H) 7.51 (broad s, 2H) 7.58 (d, J=2.0 Hz, 1H) 7.70 (d, J=9.0 Hz, 1H) 8.03 (s, 1H) 8.44 (d, J=6.8 Hz, 1H).

### Example 1c

### 1-[2-(morpholin-4-yl)ethyl]-3-(6-sulphanyl-1,3-benzothiazol-2-yl)urea

The compound can be prepared in the following way:

A solution of 11 mg of potassium dihydrogen phosphate in 2.3 ml of water is added to a suspension of 900 mg of 2-({[2-(morpholin-4-yl)ethyl]carbamoyl}amino)-1,3-benzothiazol-6-yl thiocyanate in 35 ml of ethanol at 20° C., followed by 1.1 g of DL-dithiothreitol. The white suspension is stirred for 18 h at reflux. The reaction mixture is cooled to 20° C., then 30 ml of water are added and the resulting mixture is stirred for 15 minutes. The precipitate formed is spin-filter-dried and then washed with large volumes of water. 633 mg of 1-[2-(morpholin-4-yl)ethyl]-3-(6-sulphanyl-1,3-benzothiazol-2-yl)urea are thus obtained in the form of a white solid.

MS: method B; [M+H]⁺: m/z=339; [M−H]⁻: m/z=337; Tr=2.31 min.

### Example 1d

### 2-({[2-(morpholin-4-yl)ethyl]carbamoyl}amino)-1,3-benzothiazol-6-yl thiocyanate

The compound can be prepared in the following way:

0.44 ml of 2-morpholin-4-ylethanamine is added, at 20° C., to a solution of 1 g of phenyl (6-thiocyanato-1,3-benzothiazol-2-yl)carbamate in 30 ml of tetrahydrofuran. The reaction medium is kept stirring at 20° C. for 24 hours and then concentrated by evaporation under reduced pressure. The residue obtained is chromatographed on a Merck 70 g cartridge (solid deposit; elution with a gradient of dichloromethane then 90/10 dichloromethane/methanol). 902 mg of 2-({[2-(morpholin-4-yl)ethyl]carbamoyl}amino)-1,3-benzothiazol-6-yl thiocyanate are thus obtained in the form of a colourless foam.

MS: method A; [M+H]⁺: m/z=364; Tr=0.99 min.

### Example 1e

### phenyl (6-thiocyanato-1,3-benzothiazol-2-yl)carbamate

The compound can be prepared in the following way: 7.5 g of phenyl chlorocarbonate and then 4.05 g of sodium hydrogen carbonate and 9.4 ml of water are added, at 20° C., to a solution of 2.5 g of 2-amino-1,3-benzothiazol-6-yl thiocyanate (commercial product) in 94 ml of tetrahydrofuran. The reaction medium is stirred at 20° C. for 20 hours and then extracted with twice 150 ml of ethyl acetate. The organic phases are combined and then washed three times with 50 ml of a saturated aqueous solution of sodium hydrogen carbonate. The organic phase obtained is dried over magnesium sulphate and then concentrated to dryness under reduced pressure. The residue thus obtained is taken up with 50 ml of water and the resulting product is then spin-filter-dried, and dried under vacuum at 20° C. 3.45 g of phenyl (6-thiocyanato-1,3-benzothiazol-2-yl)carbamate are thus obtained in the form of a pale yellow solid.

MS: method B; [M+H]⁺: m/z=328; [M−H]⁻: m/z=326; Tr=3.89 min.

### Example 2

### 6-{[6-(4-fluorophenyl)imidazo[1,2-a]pyridin-3-yl]sulphanyl}-1,3-benzothiazol-2-amine

### Example 2a

### 6-{[6-(4-fluorophenyl)imidazo[1,2-a]pyridin-3-yl]sulphanyl}-1,3-benzothiazol-2-amine

The compound can be prepared in the following way:

197 mg of potassium thiocyanate are added, in a single step, to a solution of 170 mg of 4-{[6-(4-fluorophenyl)imidazo[1,2-a]pyridin-3-yl]sulphanyl}aniline in 10 ml of glacial acetic acid. After stirring for approximately 20 minutes, 0.026 ml of bromine diluted in 1 ml of glacial acetic acid are run in, dropwise, while maintaining the temperature at around 20° C. The reaction mixture is stirred for approximately 18 hours at a temperature in the region of 20° C. and is then poured into 30 ml of water. The pH is brought to around 11 by adding 10N sodium hydroxide. The aqueous phase is extracted with twice 10 ml of dichloromethane and the organic phase thus obtained is washed with water, dried over magnesium sulphate, filtered, and concentrated by evaporation under reduced pressure. 164 mg of 6-{[6-(4-fluorophenyl)imidazo[1,2-a]pyridin-3-yl]sulphanyl}-1,3-benzothiazol-2-amine are thus obtained in the form of a yellow solid.

Melting point: 258° C. (Köfler bench).

MS: method A; [M+H]⁻: m/z=391; [M+H]⁺: m/z=393; [M+2H]²⁺: m/z=197; [M+CH₃CN+2H]²⁺: m/z=217 (base peak); Tr=0.70 min.

¹H NMR (400 MHz, DMSO-d₆) δ ppm 7.12 (dd, J=8.3, 2.1 Hz, 1H) 7.23 (d, J=8.3 Hz, 1H) 7.31 (t, J=8.4 Hz, 2H) 7.50 (broad s, 2H) 7.64 (d, J=1.7 Hz, 1H) 7.68-7.75 (m, 3H) 7.80 (dd, J=9.3, 1 Hz, 1H) 8.07 (s, 1H) 8.56 (broad s, 1H).

### Example 2b

### 4-{[6-(4-fluorophenyl)imidazo[1,2-a]pyridin-3-yl]sulphanyl}aniline

The compound can be prepared in the following way:

A solution of 200 mg of N-(4-{[6-(4-fluorophenyl)imidazo[1,2-a]pyridin-3-yl]sulphanyl}phenyl)acetamide, 15 ml of ethanol and 1 ml of hydrochloric acid (37% by volume) is brought to reflux for 6 hours. 0.5 ml of hydrochloric acid (37% by volume) is, then added and the reaction medium is again brought to reflux for 5 hours and then left to stir at a temperature in the region of 20° C. for 18 hours. The medium is then poured into 50 ml of a saturated aqueous solution of sodium hydrogen carbonate, and the aqueous phase is extracted with 3 times 20 ml of dichloromethane. The organic phase is washed with 3 times 10 ml of water, dried over magnesium sulphate, filtered, and concentrated by evaporation under reduced pressure. 173 mg of 4-{[6-(4-fluorophenyl)imidazo[1,2-a]pyridin-3-yl]sulphanyl}aniline are thus obtained in the form of a beige solid.

MS: method A; [M+H]⁺: m/z=336; Tr=0.70 min.

### Example 2c

### N-(4-{[6-(4-fluorophenyl)imidazo[1,2-a]pyridin-3-yl]sulphanyl}-phenyl)acetamide

The compound can be prepared in the following way:

3.7 g of 3-bromo-6-(4-fluorophenyl)imidazo[1,2-a]pyridine, 3.2 g of N-(4-sulphanylphenyl)acetamide (commercial product), 4.4 g of potassium carbonate and 62 ml of dimethyl sulphoxide are charged to a sealed glass tube. The medium is microwave-heated at 190° C. for 15 minutes. After a return to a temperature in the region of 20° C., the medium is poured into 800 ml of water and ice and extracted with twice 250 ml of ethyl acetate and the combined organic extracts are dried over magnesium sulphate, filtered, and concentrated to dryness under reduced pressure. The evaporation residue is chromatographed, under an argon pressure, on silica gel (eluent: 97/3 ethyl acetate/methanol) and makes it possible to isolate a solid which is triturated from diisopropyl ether. 700 mg of N-(4-{[6-(4-fluorophenyl)-imidazo[1,2-a]pyridin-3-yl]sulphanyl}phenyl)acetamide are thus obtained in the form of a brown solid.

MS: method B; [M+H]⁺: m/z=378; [M−H]⁻: m/z=376; [M+HCO₂H—H]⁻: m/z=422; Tr=3.25 min.

### Example 2d

### 3-bromo-6-(4-fluorophenyl)imidazo[1,2-a]pyridine

The compound can be prepared in the following way:

A solution of 1 ml of bromine in 40 ml of water is run, dropwise, into a solution of 3.61 g of 6-(4-fluorophenyl)imidazo[1,2-a]pyridine in 65 ml of ethanol. After stirring for 2.5 hours at a temperature in the region of 20° C., the reaction medium is poured into a saturated aqueous solution of sodium hydrogen carbonate, and the aqueous phase is extracted with 3 times 20 ml of ethyl acetate. The organic phase is concentrated by evaporation under reduced pressure. 3.1 g of 3-bromo-6-(4-fluorophenyl)imidazo[1,2-a]pyridine are thus obtained in the form of a red solid.

MS: method A; [M+H]⁺: m/z=291; Tr=0.71 min.

### Example 2e

### 6-(4-fluorophenyl)imidazo[1,2-a]pyridine

The compound can be prepared in the following way: 1.76 g of 4-fluorophenylboronic acid are added to a mixture of 3.44 g of 6-iodoimidazo[1,2-a]pyridine, 110 ml of dioxane, 132 mg of tetrakis(triphenylphosphine)palladium and 2.1 g of sodium hydrogen carbonate in solution in 65 ml of water. The reaction medium is heated at 90° C. for 1.5 hours. 0.3 g of 4-fluorophenylboronic acid is then added and the medium is again brought to 80° C. for 1 hour. After cooling, the reaction medium is poured into 350 ml of water, and 150 ml of ethyl acetate are added. The aqueous phase is extracted with twice 100 ml of ethyl acetate, and the combined organic phases are dried over magnesium sulphate, filtered, and concentrated by evaporation under reduced pressure. 3 g of 6-(4-fluorophenyl)imidazo[1,2-a]pyridine are thus obtained in the form of a red solid.

MS: method A; [M+H]⁺: m/z=213; Tr=0.42 min.

### Example 2f

### 6-iodoimidazo[1,2-a]pyridine

The compound can be prepared as described by C. Enguehard et al., Helvetica Chimica Acta (2001), 84, 3610-3614.

### Example 3

### N-(6-{[6-(4-fluorophenyl)imidazo[1,2-a]pyridin-3-yl]sulphanyl}-1,3-benzothiazol-2-yl)cyclopropanecarboxamide

The compound can be prepared in the following way:

0.033 ml of cyclopropanecarbonyl chloride is added to a suspension of 130 mg of 6-{[6-(4-fluorophenyl)imidazo[1,2-a]pyridin-3-yl]sulphanyl}-1,3-benzothiazol-2-amine and 3 ml of pyridine. After stirring overnight at a temperature in the region of 20° C., 0.037 ml of cyclopropanecarbonyl chloride is added. After stirring overnight at a temperature in the region of 20° C., 10 ml of water are added and the precipitate obtained is spin-filter-dried, washed with 3 times 10 ml of water and 3 times 10 ml of ethanol, and oven-dried at 50° C. under reduced pressure. 119 mg of N-(6-{[6-(4-fluorophenyl)-imidazo[1,2-a]pyridin-3-yl]sulphanyl}-1,3-benzothiazol-2-yl)cyclopropane-carboxamide are thus obtained in the form of a white solid.

Melting point: 265° C. (Büchi).

MS: method A; [M+H]⁺: m/z=461; [M−H]⁻: m/z=459; Tr=0.91 min.

¹H NMR (400 MHz, DMSO-d₆) δ ppm 0.84-1.03 (m, 4H) 1.89-2.03 (m, 1H) 7.20 (d, J=8.5 Hz, 1H) 7.30 (t, J=8.5 Hz, 2H) 7.61 (d, J=8.5 Hz, 1H) 7.69 (m, 2H) 7.75 (d, J=9.8 Hz, 1H) 7.82 (d, J=9.8 Hz, 1H) 7.88 (s, 1H) 8.12 (s, 1H) 8.55 (s, 1H) 12.59 (m, 1H).

### Example 4

### N-(6-{[6-(4-fluorophenyl)imidazo[1,2-a]pyridin-3-yl]sulphanyl}-1,3-benzothiazol-2-yl)acetamide

The compound can be prepared in the following way:

A solution of 56 mg of 6-{[6-(4-fluorophenyl)imidazo[1,2-a]pyridin-3-yl]sulphanyl}-1,3-benzothiazol-2-amine, 1.2 ml of pyridine and 1.2 ml of acetic anhydride is brought to reflux for 2 hours. The reaction medium is then concentrated by evaporation under reduced pressure and the solid residue is taken up in 2 ml of methanol, spin-filter-dried, washed with 3 times 1 ml of methanol and then dried. 18 mg of N-(6-{[6-(4-fluorophenyl)imidazo[1,2-a]pyridin-3-yl]sulphanyl}-1,3-benzothiazol-2-yl)acetamide are thus obtained in the form of a brown solid.

MS: method A; [M+H]⁺: m/z=435; [M−H]⁻: m/z=433; Tr=0.80 min.

¹H NMR (400 MHz, DMSO-d₆) δ ppm 2.17 (s, 3H) 7.21 (dd, J=8.7, 1.1 Hz, 1H) 7.30 (t, J=8.7 Hz, 2H) 7.62 (d, J=8.3 Hz, 1H) 7.65-7.73 (m, 2H) 7.73-7.78 (m, 1H) 7.80-7.86 (m, 1H) 7.88 (s, 1H) 8.12 (s, 1H) 8.55 (s, 1H) 12.30 (br. s., 1H).

### Example 5

### 1-(6-{[6-(4-fluorophenyl)imidazo[1,2-a]pyridin-3-yl]sulphanyl}-1,3-benzothiazol-2-yl)-3-(2-methoxyethyl)urea

### Example 5a

### 1-(6-{[6-(4-fluorophenyl)imidazo[1,2-a]pyridin-3-yl]sulphanyl}-1,3-benzothiazol-2-yl)-3-(2-methoxyethyl)urea

The compound can be prepared in the following way:

18.6 μl of 2-methoxyethanamine are added to a suspension of 0.1 g of phenyl (6-{[6-(4-fluorophenyl)imidazo[1,2-a]pyridin-3-yl]sulphanyl}-1,3-benzothiazol-2-yl)carbamate in 3 ml of tetrahydrofuran. After stirring for 5 hours at a temperature in the region of 20° C., 18 μl of 2-methoxyethanamine in solution in 2 ml of tetrahydrofuran are added and the reaction mixture is stirred overnight at a temperature in the region, of 20° C. The precipitate formed is spin-filter-dried, washed with twice 3 ml of methanol, and dried. 70 mg of 0.13 g of 1-(6-{[6-(4-fluorophenyl)imidazo[1,2-a]pyridin-3-yl]sulphanyl}-1,3-benzothiazol-2-yl)-3-(2-methoxyethyl)urea are thus obtained in the form of a white solid.

Melting point>260° C. (Köfler bench)

MS: method A; [M+H]⁺: m/z=494; [M−H]⁻: m/z=492; Tr=0.82 min.

¹H NMR (400 MHz, DMSO-d₆) δ ppm 2.48-2.53 (m partially masked, 2H) 3.27 (broad s, 3H) 3.40 (m, 2H) 6.80 (m, 1H) 7.16 (broad d, J=8.5 Hz, 1H) 7.30 (t, J=8.4 Hz, 2H) 7.51 (d, J=8.5 Hz, 1H) 7.67-7.77 (m, 3H) 7.80-7.85 (m, 2H) 8.11 (s, 1H) 8.56 (broad s, 1H) 10.60 (broad m, 1H).

### Example 5b

### phenyl (6-{[6-(4-fluorophenyl)imidazo[1,2-a]pyridin-3-yl]sulphanyl}-1,3-benzothiazol-2-yl)carbamate

The compound can be prepared in the following way:

0.257 ml of phenyl chlorocarbonate and then 171 mg of sodium hydrogen carbonate in 0.5 ml of water are added to a suspension of 200 mg of 6-{[6-(4-fluorophenyl)imidazo[1,2-a]pyridin-3-yl]sulphanyl}-1,3-benzothiazol-2-amine in 5 ml of tetrahydrofuran. The mixture is stirred at a temperature in the region of 20° C. for approximately 24 hours. 0.15 ml of phenyl chlorocarbonate and 0.1 g of sodium hydrogen carbonate in 0.3 ml of water are then added. After stirring for 2 hours, a further 0.05 ml of phenyl chlorocarbonate and 0.05 g of sodium hydrogen carbonate in 0.3 ml of water are added. After stirring for 2 hours, the medium is poured into 10 ml of water and the precipitate formed is spin-filter-dried, washed with twice 5 ml of water then with twice 5 ml of ethyl acetate, and air-dried. 138 mg of phenyl (6-{[6-(4-fluorophenyl)imidazo[1,2-a]pyridin-3-yl]sulphanyl}-1,3-benzothiazol-2-yl)carbamate are thus obtained in the form of a white powder.

MS: method B; [M+H]⁺: m/z=513; [M−H]⁻: m/z=511; Tr=4.25 min.

### Example 6

### 1-(6-{[6-(4-fluorophenyl)imidazo[1,2-a]pyridin-3-yl]sulphanyl}-1,3-benzothiazol-2-yl)-3-[2-(morpholin-4-yl)ethyl]urea

The compound can be prepared as in Example 5a, but using 0.15 g of phenyl (6-{[6-(4-fluorophenyl)imidazo[1,2-a]pyridin-3-yl]sulphanyl}-1,3-benzothiazol-2-yl)carbamate, 46 μl of 2-(morpholin-4-yl)ethanamine and 5 ml of tetrahydrofuran. 42 mg of 1-(6-{[6-(4-fluorophenyl)imidazo[1,2-a]pyridin-3-yl]sulphanyl}-1,3-benzothiazol-2-yl)-3-[2-(morpholin-4-yl)ethyl]urea are thus obtained in the form of a white powder.

MS: method A; [M+H]⁺: m/z=549; [M−H]⁻: m/z=547; Tr=0.65 min.

¹H NMR (400 MHz, DMSO-d₆) δ ppm 2.39 (m, 6H) 3.31 (m, 2H) 3.54-3.61 (m partially masked, 4H) 6.71-6.78 (m, 1H) 7.18 (broad d, J=8.5 Hz, 1H) 7.30 (t, J=8.4 Hz, 2H) 7.50 (d, J=8.5 Hz, 1H) 7.66-7.76 (m, 3H) 7.82 (m, 2H) 8.10 (s, 1H) 8.55 (broad s, 1H) 10.60 (broad m, 1H).

### Example 7

### N-(6-{[6-(1-methyl-1H-pyrazol-4-yl)imidazo[1,2-a]pyridin-3-yl]sulphanyl}-1,3-benzothiazol-2-yl)cyclopropanecarboxamide

### Example 7a

### N-(6-{[6-(1-methyl-1H-pyrazol-4-yl)imidazo[1,2-a]pyridin-3-yl]sulphanyl}-1,3-benzothiazol-2-yl)cyclopropanecarboxamide

The compound can be prepared as in Example 1b, but using 0.57 g of 3-bromo-6-(1-methyl-1H-pyrazol-4-yl)imidazo[1,2-a]pyridine, 0.618 g of (6-sulphanyl-1,3-benzothiazol-2-yl)cyclopropanecarboxamide, 0.852 g of potassium carbonate and 5 ml of dimethyl sulphoxide. 0.28 g of N-(6-{[6-(1-methyl-1H-pyrazol-4-yl)imidazo[1,2-a]pyridin-3-yl]sulphanyl}-1,3-benzothiazol-2-yl)cyclopropanecarboxamide is thus obtained in the form of a pale yellow solid.

MS: method A; [M+H]⁺ m/z=447; [M−H]⁻ m/z=445; Tr=0.64 min.

¹H NMR (400 MHz, DMSO-d₆) δ ppm 0.90-0.96 (m, 4H) 1.88-2.01 (m, 1H) 3.85 (s, 3H) 7.22 (dd, J=8.4, 1.5 Hz, 1H) 7.62 (d, J=8.4 Hz, 1H) 7.67 (dd, J=9.0, 1.5 Hz, 1H) 7.75 (d, J=9.0 Hz, 1H) 7.88-7.92 (m, 2H) 8.04 (s, 1H) 8.23 (s, 1H) 8.53 (s, 1H) 12.59 (br. s., 1H).

### Example 7b

### (6-sulphanyl-1,3-benzothiazol-2-yl)cyclopropanecarboxamide

The compound can be prepared in the following way:

A solution of 33.6 mg of potassium dihydrogen phosphate in 8 ml of water at 20° C., followed by 3.2 g of DL-dithiothreitol, are added to a suspension of 2 g of (6-thiocyanato-1,3-benzothiazol-2-yl)cyclopropanecarboxamide and 70 ml of ethanol. The reaction medium is stirred at reflux for 5 h and then brought back to a temperature in the region of 20° C. 400 ml of water are then added, and the precipitate formed is filtered off through sintered glass, washed thoroughly with water, spin-filter-dried, and then dried. 1.5 g of (6-sulphanyl-1,3-benzothiazol-2-yl)cyclopropanecarboxamide are thus obtained in the form of a pale yellow solid.

MS: method B; [M+H]⁺ m/z=251; [M−H]⁻ m/z=249; Tr=3.77 min.

### Example 7c

### (6-thiocyanato-1,3-benzothiazol-2-yl)cyclopropanecarboxamide

The compound can be prepared in the following way:

5.3 ml of cyclopropanecarbonyl chloride are added to a solution of 10 g of 2-amino-1,3-benzothiazol-6-yl thiocyanate (commercial product) and 100 ml of pyridine, while maintaining the temperature in the region of 20° C. The reaction medium is stirred for 4 hours and then 500 ml of water are added. The precipitate formed is filtered off through sintered glass, washed thoroughly with water, spin-filter-dried, and then dried. 13 g of (6-thiocyanato-1,3-benzothiazol-2-yl)cyclopropanecarboxamide are thus obtained in the form of a pale yellow solid, said compound being used as it is in the subsequent stages.

### Example 7d

### 3-bromo-6-(1-methyl-1H-pyrazol-4-yl)imidazo[1,2-a]pyridine

The compound can be prepared as in Example 2d, but using 1.5 g of 6-(1-methyl-1H-pyrazol-4-yl)imidazo[1,2-a]pyridine, 0.46 ml of bromine, 20 ml of water and 30 ml of ethanol. 1.72 g of 3-bromo-6-(1-methyl-1H-pyrazol-4-yl)imidazo[1,2-a]pyridine are thus obtained in the form of a cream solid.

MS: method A; [M+H]⁺ m/z=277; Tr=0.35 min.

### Example 7e

### 6-(1-methyl-1H-pyrazol-4-yl)imidazo[1,2-a]pyridine

The compound can be prepared as in Example 2e, but using 3 g of 6-iodoimidazo[1,2-a]pyridine, 27 ml of dimethylformamide, 125 mg of tetrakis(triphenylphosphine)palladium, 1.4 g of sodium hydrogen carbonate in solution in 18 ml of water and 2.7 g of (1-methyl-1H-pyrazol-4-yl)boronic acid. 1.5 g of 6-(1-methyl-1H-pyrazol-4-yl)imidazo[1,2-a]pyridine are thus obtained.

MS: method B; [M+H]⁺ m/z=199; Tr=0.5 min.

### Example 8

### N-(6-{[6-(1H-pyrazol-4-yl)imidazo[1,2-a]pyridin-3-yl]sulphanyl}-1,3-benzothiazol-2-yl)cyclopropanecarboxamide

### Example 8a

### N-(6-{[6-(1H-pyrazol-4-yl)imidazo[1,2-a]pyridin-3-yl]sulphanyl}-1,3-benzothiazol-2-yl)cyclopropanecarboxamide

The compound can be prepared as in Example 1b, but using 0.331 g of 3-bromo-6-(1H-pyrazol-4-yl)imidazo[1,2-a]pyridine, 0.252 g of (6-sulphanyl-1,3-benzothiazol-2-yl)cyclopropanecarboxamide, 0.278 g of potassium carbonate and 3.3 ml of dimethyl sulphoxide. 0.025 g of N-(6-{[6-(1H-pyrazol-4-yl)imidazo[1,2-a]pyridin-3-yl]sulphanyl}-1,3-benzothiazol-2-yl)cyclopropane-carboxamide is thus obtained in the form of a cream solid.

MS: method B; [M+H]⁺ m/z=433; [M−H]⁻ m/z=431; Tr=2.82 min.

¹H NMR (400 MHz, DMSO-d₆) δ ppm 0.88-1.00 (m, 4H) 1.93-2.03 (m, 1 H) 7.50 (dd, J=8.5, 2.0 Hz, 1H) 7.69 (d, J=8.5 Hz, 1H) 8.03 (d, J=9.5 Hz, 1H) 8.08 (d, J=2 Hz, 1H) 8.25 (d, J=9.5 Hz, 1H) 8.30 (s, 2H) 8.66 (s, 1H) 8.86 (s, 1H) 12.66 (s, 1H).

### Example 8b

### 3-bromo-6-(1H-pyrazol-4-yl)imidazo[1,2-a]pyridine

The compound can be prepared as in Example 2d, but using 0.789 g of 6-(1H-pyrazol-4-yl)imidazo[1,2-a]pyridine, 0.263 ml of bromine, 10 ml of water and 16 ml of ethanol. 1 g of 3-bromo-6-(1H-pyrazol-4-yl)imidazo[1,2-a]-pyridine is thus obtained in the form of a brown solid.

MS: method B; [M+H]⁺ m/z=263; [M−H]⁻ m/z=261; Tr=0.81 min.

### Example 8c

### 6-(1H-pyrazol-4-yl)imidazo[1,2-a]pyridine

The compound can be prepared as in Example 2e, but using 2 g of 6-iodoimidazo[1,2-a]pyridine, 18 ml of dimethylformamide, 85 mg of tetrakis(triphenylphosphine)palladium, 0.84 g of sodium hydrogen carbonate in solution in 12 ml of water and 0.96 g of (1H-pyrazol-4-yl)boronic acid. 0.789 g of 6-(1H-pyrazol-4-yl)imidazo[1,2-a]pyridine is thus obtained.

MS: method A; [M+H]⁺ m/z=185; Tr=0.16 min.

### Example 9

### N-(6-{[6-(3-fluorophenyl)imidazo[1,2-a]pyridin-3-yl]sulphanyl}-1,3-benzothiazol-2-yl)cyclopropanecarboxamide

### Example 9a

### N-(6-{[6-(3-fluorophenyl)imidazo[1,2-a]pyridin-3-yl]sulphanyl}-1,3-benzothiazol-2-yl)cyclopropanecarboxamide

The compound can be prepared as in Example 1b, but using 0.9 g of 3-bromo-6-(3-fluorophenyl)imidazo[1,2-a]pyridine, 0.9 g of (6-sulphanyl-1,3-benzothiazol-2-yl)cyclopropanecarboxamide, 0.910 g of potassium carbonate and 9 ml of dimethyl sulphoxide. 0.168 g of N-(6-{[6-(3-fluorophenyl)-imidazo[1,2-a]pyridin-3-yl]sulphanyl}-1,3-benzothiazol-2-yl)cyclopropane-carboxamide is thus obtained in the form of a yellow solid.

Melting point>260° C. (Köfler bench)

MS: method B; [M+H]⁺ m/z=461; [M−H]⁻ m/z=459; Tr=3.91 min

¹H NMR (400 MHz, DMSO-d₆) δ ppm 0.89-0.95 (m, 4H) 1.93-1.98 (m, 1H) 7.19-7.26 (m, 2H) 7.47-7.57 (m, 3H) 7.62 (d, J=8.8 Hz, 1H) 7.79 (dd, J=9.3, 2.0 Hz, 1H) 7.84 (d, J=9.3 Hz, 1H) 7.90 (d, J=1.5 Hz, 1H) 8.13 (s, 1 H) 8.63 (s, 1H) 12.59 (br. s., 1H).

### Example 9b

### 3-bromo-6-(3-fluorophenyl)imidazo[1,2-a]pyridine

The compound can be prepared as in Example 2d, but using 1.7 g of 6-(3-fluorophenyl)imidazo[1,2-a]pyridine, 0.42 ml of bromine, 20 ml of water and 35 ml of ethanol. 1 g of 3-bromo-6-(3-fluorophenyl)imidazo[1,2-a]pyridine is thus obtained in the form of a brown solid.

MS: method A; [M+H]⁺ m/z=291; Tr=0.74 min.

### Example 9c

### 6-(3-fluorophenyl)imidazo[1,2-a]pyridine

The compound can be prepared as in Example 2e, but using 2 g of 6-iodoimidazo[1,2-a]pyridine, 35 ml of dimethylformamide, 83 mg of tetrakis(triphenylphosphine)palladium, 1.64 g of sodium hydrogen carbonate in solution in 23 ml of water, and 1.23 g of 3-fluorophenylboronic acid. 1.7 g of 6-(3-fluorophenyl)imidazo[1,2-a]pyridine are thus obtained.

MS: method A; [M+H]⁺ m/z=213; Tr=0.41 min.

### Example 10

### N-(6-{(6-[((3-fluoro-4-methyl)phenyl)imidazo[1,2-a]pyridin-3-yl]sulphanyl}-1,3-benzothiazol-2-yl)cyclopropanecarboxamide

### Example 10a

### N-(6-{[6-((3-fluoro-4-methyl)phenyl)imidazo[1,2-a]pyridin-3-yl]sulphanyl}-1,3-benzothiazol-2-yl)cyclopropanecarboxamide

The compound can be prepared as in Example 1b, but using 1.22 g of 3-bromo-6-((3-fluoro-4-methyl)phenyl)imidazo[1,2-a]pyridine, 1.03 g of (6-sulphanyl-1,3-benzothiazol-2-yl)cyclopropanecarboxamide, 1.3 g of potassium carbonate and 9 ml of dimethyl sulphoxide. 0.32 g of N-(6-{[6-((3-fluoro-4-methyl)phenyl)imidazo[1,2-a]pyridin-3-yl]sulphanyl}-1,3-benzothiazol-2-yl)cyclopropanecarboxamide is thus obtained in the form of a yellow solid.

Melting point>260° C. (Köfler bench)

MS: method A; [M+H]⁺ m/z=473; [M−H]⁻ m/z=475; Tr=0.99 min.

¹H NMR (400 MHz, DMSO-d₆) δ ppm 0.89-0.97 (m, 4H) 1.92-2.00 (m, 1H) 2.26 (s, 3H) 7.22 (dd, J=8.3, 1.5 Hz, 1H) 7.33-7.42 (m, 2H) 7.47 (d, J=10.7 Hz, 1H) 7.62 (d, J=8.3 Hz, 1H) 7.77 (dd, J=9.3 Hz, 1H) 7.82 (d, J=9.3 Hz, 1H) 7.90 (d, J=1.5 Hz, 1H) 8.12 (s, 1H) 8.58 (s, 1H) 12.60 (br. s., 1H).

### Example 10b

### 3-bromo-6-((3-fluoro-4-methyl)phenyl)imidazo[1,2-a]pyridine

The compound can be prepared as in Example 2d, but using 1.7 g of 6-((3-fluoro-4-methyl)phenyl)imidazo[1,2-a]pyridine, 0.37 ml of bromine; 15 ml of water and 30 ml of ethanol. 1.22 g of 3-bromo-6-((3-fluoro-4-methyl)phenyl)imidazo[1,2-a]pyridine are thus obtained in the form of a grey solid.

MS: method A; [M+H]⁺ m/z=305; Tr=0.86 min.

### Example 10c

### 6-((3-fluoro-4-methyl)phenyl)imidazo[1,2-a]pyridine

The compound can be prepared as in Example 2e, but using 2.1 g of 6-iodoimidazo[1,2-a]pyridine, 30 ml of dimethylformamide, 85 mg of tetrakis(triphenylphosphine)palladium, 1.73 g of sodium hydrogen carbonate in solution in 23 ml of water and 1.38 g of (3-fluoro-4-methyl)phenylboronic acid. 1.7 g of 6-((3-fluoro-4-methyl)phenyl)imidazo[1,2-a]pyridine are thus obtained in the form of a brown solid.

MS: method A; [M+H]⁺ m/z=227; Tr=0.52 min.

### Example 11

### tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}piperidine-1-carboxylate

### Example 11a

### tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}piperidine-1-carboxylate

The compound can be prepared in the following way:

340 mg of tert-butyl 4-[4-(3-bromoimidazo[1,2-a]pyridin-6-yl)-1H-pyrazol-1-yl]piperidine-1-carboxylate, 210 mg of (6-sulphanyl-1,3-benzothiazol-2-yl)cyclopropanecarboxamide, 250 μl of N,N-diisopropylethylamine, 104 mg of tris(dibenzylideneacetone)dipalladium(0), 132 mg of 4,5-bis(diphenyl-phosphino)-9,9-dimethylxanthene and 10 ml of 1,4-dioxane are charged to a sealed glass tube. After sparging with argon in the reaction medium for 5 minutes, the medium is microwave-heated at 160° C. for 25 minutes. After a return to a temperature in the region of 20° C., the medium is diluted with 25 ml of a 95/5, by volume, mixture of dichloromethane/methanol and the organic phase is then washed with twice 30 ml of distilled water, dried over magnesium sulphate, filtered, and concentrated to dryness under reduced pressure. The evaporation residue is chromatographed, under an argon pressure, on silica gel (eluent: dichloromethane/methanol 96/4 by volume). 217 mg of tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}piperidine-1-carboxylate are thus obtained in the form of a white solid.

Melting point: 247° C. (Kôfler bench)

MS: method A; [M+H]⁺ m/z=616; [M−H]⁻ m/z=445; Tr=0.91 min.

¹H NMR (400 MHz, DMSO-d6) δ ppm 0.83 to 1.02 (m, 4H); 1.42 (s, 9H); 1.69 to 1.85 (m, 2H); 1.88 to 2.09 (m, 3H); 2.80 to 3.01 (m, 2H); 4.04 (d, J=14.4 Hz, 2H); 4.27 to 4.43 (m, 1H); 7.21 (dd, J=2.0 and 8.6 Hz, 1H); 7.62 (d, J=8.3 Hz, 1H); 7.70 (dd, J=1.7 and 9.3 Hz, 1H); 7.75 (dd, J=1.0 and 9.3 Hz, 1H); 7.90 (d, J=2.0 Hz, 1H); 7.95 (s, 1H); 8.03 (s, 1H); 8.38 (s, 1H); 8.57 (dd, J=1.0 and 1.7 Hz, 1H); 12.59 (broad s, 1H).

### Example 11b

### tert-butyl 4-[4-(3-bromoimidazo[1,2-a]pyridin-6-yl)-1H-pyrazol-1-yl]piperidine-1-carboxylate

The compound can be prepared in the following way:

A mixture of 860 mg of tert-butyl 4-[4-(imidazo[1,2-a]pyridin-6-yl)-1H-pyrazol-1-yl]piperidine-1-carboxylate, 80 ml of chloroform and 417 mg of N-bromo-succinimide is brought to reflux overnight. The medium is cooled to a temperature in the region of 20° C. and then concentrated by evaporation under reduced pressure. The residue isolated is chromatographed, under an argon pressure, on silica gel (eluent: ethyl acetate/methanol 80/20 by volume). 1.046 g of tert-butyl 4-[4-(3-bromoimidazo[1,2-a]pyridin-6-yl)-1H-pyrazol-1-yl]piperidine-1-carboxylate are thus obtained in the form of an amber gum, used as it is in the subsequent stages.

MS: method A; [M+H]⁺ m/z=446; Tr=0.76 min.

¹H NMR (400 MHz, DMSO-d6) δ ppm: 0.83 to 1.02 (m, 4H); 1.42 (s, 9H); 1.69 to 1.85 (m, 2H); 1.88 to 2.09 (m, 3H); 2.80 to 3.01 (m, 2H); 4.04 (d, J=14.4 Hz, 2H); 4.27 to 4.43 (m, 1H); 7.21 (dd, J=2.0 and 8.6 Hz, 1H); 7.62 (d, J=8.3 Hz, 1H); 7.70 (dd, J=1.7 and 9.3 Hz, 1H); 7.75 (dd, J=1.0 and 9.3 Hz, 1H); 7.90 (d, J=2.0 Hz, 1H); 7.95 (s, 1H); 8.03 (s, 1H); 8.38 (s, 1H); 8.57 (dd, J=1.0 and 1.7 Hz, 1H); 12.59 (broad s, 1H).

### Example 11c

### tert-butyl 4-[4-(imidazo[1,2-a]pyridin-6-yl)-1H-pyrazol-1-yl]piperidine-1-carboxylate

The compound can be prepared in the following way:

A solution of 1.1 g of 6-iodoimidazo[1,2-a]pyridine hydrochloride, 45 ml of 1,2-dimethoxyethane, 3.2 ml of sodium hydroxide (1N aqueous solution) and tert-butyl 4-[4-(4,4,5,5-tetramethyl-1,3,2-dioxaborolan-2-yl)-pyrazol-1-yl]-piperidine-1-carboxylate is stirred for 30 minutes at a temperature in the region of 20° C. 138 mg of dichlorobis(triphenylphosphine)palladium are then added and the reaction medium is brought to 65° C. for 30 minutes and then stirred for 16 hours at a temperature in the region of 20° C. The reaction medium is then poured into 450 ml of distilled water and extracted with 4 times 60 ml of dichloromethane. The combined organic extracts are washed with 60 ml of distilled water, dried over magnesium sulphate, filtered, and concentrated to dryness under reduced pressure. The residue obtained is chromatographed, under an argon pressure, on silica gel (eluent: dichloromethane/methanol 96/4 by volume). 865 mg of tert-butyl 4-[4-(imidazo[1,2-a]pyridin-6-yl)-1H-pyrazol-1-yl]piperidine-1-carboxylate are thus obtained in the form of a yellow oil.

MS: method A; [M+H]⁺ m/z=368; Tr=0.60 min.

¹H NMR (400 MHz, DMSO-d6) δ ppm: 1.43 (s, 9H); 1.81 (qd, J=4.3 and 12.2 Hz, 2H); 2.05 (dd, J=2.0 and 12.0 Hz, 2H); 2.88 to 3.00 (m, 2H); 4.02 to 4.06 (m, 2H); 4.34 to 4.44 (m, 1H); 7.45 to 7.52 (m, 1H); 7.53 to 7.60 (m, 2H); 7.86 (s, 1H); 7.90 (s, 1H); 8.29 (s, 1H); 8.80 (s, 1H).

### Example 11d

### tert-butyl 4-[4-(4,4,5,5-tetramethyl-1,3,2-dioxaborolan-2-yl)-pyrazol-1-yl]-piperidine-1-carboxylate

The compound can be prepared as described in patent WO2007/066187, page 34.

### Example 12

### N-[6-({6-[1-(piperidin-4-yl)1H-pyrazol-4-yl]imidazo[1,2-a]pyridin-3-yl}sulphanyl)-1,3-benzothiazol-2-yl]cyclopropanecarboxamide

The compound can be prepared in the following way:

A mixture of 176 mg of tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}-piperidine-1-carboxylate and 2.6 ml of hydrochloric acid in 1,4-dioxane (4M solution) is stirred at a temperature in the region of 20° C. for 16 hours. The medium is then concentrated to dryness by evaporation under reduced pressure, and the solid isolated is triturated with 5 ml of isopropyl ether, filtered through sintered glass, washed with three times 5 ml of isopropyl ether, spin-filter-dried, and dried under reduced pressure. The solid is again taken up in 5 ml of acetone and triturated for 5 minutes, and then filtered, washed with twice 5 ml of acetone, spin-filter-dried, and dried under reduced pressure. 160 mg of N-[6-({6-[1-(piperidin-4-yl)-1H-pyrazol-4-yl]imidazo[1,2-a]pyridin-3-yl}sulphanyl)-1,3-benzothiazol-2-yl]cyclopropanecarboxamide hydrochloride are thus obtained in the form of a beige solid.

Melting point: 230° C. tacky (Köfler bench).

MS: method A; [M+H]⁺ m/z=516; [M−H]⁻ m/z=514; Tr=0.51 min.

¹H NMR (400 MHz, DMSO-d6) δ ppm: 0.87 to 0.99 (m, 4H); 1.93 to 2.04 (m, 1H); 2.08 to 2.29 (m, 4H); 3.00 to 3.15 (m, 2H); 3.34 to 3.43 (m, J=13.7 Hz, 2H); 4.44 to 4.57 (m, 1H); 7.42 (dd, J=2.1 and 8.4 Hz, 1H); 7.67 (d, J=8.6 Hz, 1H); 7.94 to 8.04 (m, 2H); 8.10 to 8.17 (m, 2H); 8.50 (s, 1H); 8.54 (s, 1H); 8.73 to 8.89 (m, 2H); 8.96 to 9.09 (m, 1H); 12.65 (s, 1H).

### Example 13

### Pharmaceutical Composition

Tablets corresponding to the following formulation were prepared:

| | |
|---|---|
| Product of Example 1 | 0.2 g |
| Excipient for a final tablet of | 1 g |
| (excipient details: lactose, | |
| talc, starch, magnesium stearate). | |

Example 1 is taken as an example of a pharmaceutical preparation, it being possible for this preparation to be carried out, if desired, with other products in the examples in the present application.

### Pharmacological Section:

### Experimental Protocols

### I) Expression and Purification of MET, Cytoplasmic Domain

### Expression in Baculovirus:

The His-Tev-MET (956-1390) recombinant DNA in pFastBac (Invitrogen) is transfected into insect cells and, after several viral amplification steps, the final baculovirus stock is tested for the expression of the protein of interest.

After infection for 72 h at 27° C. with the recombinant virus, SF21 cell cultures are harvested by centrifugation and the cell pellets are stored at −80° C.

### Purification:

The cell pellets are resuspended in the lysis buffer (buffer A [50 mM HEPES, pH 7.5, 250 mM NaCl, 10% glycerol, 1 mM TECP]; +cocktail of protease inhibitors, Roche Diagnostics, without EDTA, ref 1873580), stirred at 4° C. until the mixture is homogeneous, and then lysed mechanically using a “Dounce” type apparatus.

After centrifugation, the lysis supernatant is incubated for 2 h at 4° C. with nickel chelate resin (His-Trap 6 Fast Flow™, GE HealthCare). After washing with 20 volumes of buffer A, the suspension is packed into a column, and the proteins are eluted with a gradient of buffer B (buffer A+290 mM imidazole).

The fractions containing the protein of interest, for the purpose of electrophoretic analysis (SDS PAGE), are combined, concentrated by ultrafiltration (10 kDa cut-off) and injected onto an exclusion chromatography column (Superdex® 200, GE HealthCare) equilibrated in buffer A.

After enzymatic cleavage of the histidine tag, the protein is reinjected onto a new IMAC nickel chelate chromatography column (His-Trap 6 Fast Flow™ GE HealthCare) equilibrated in buffer A. The fractions eluted with a gradient of buffer B and containing the protein of interest after electrophoresis (SDS PAGE) are finally combined and stored at −80° C.

For the production of autophosphorylated protein, the previous fractions are incubated for 1 h at ambient temperature after the addition of 2 mM ATP, 2 mM MgCl₂ and 4 mM Na₃VO₄. After the reaction has been stopped with 5 mM of EDTA, the reaction mixture is injected onto a HiPrep desalifying column (GE HealthCare) pre-equilibrated in buffer A+4 mM Na₃VO₄, and the fractions containing the protein of interest (SDS PAGE analysis) are combined and stored at −80° C. The degree of phosphorylation is verified by mass spectrometry (LC-MS) and by peptide mapping.

### II) Tests A and B

### A) Test A: HTRF MET Assay in 96-Well Format

MET at a final concentration of 5 nM is incubated in a final volume of 50 μl of enzymatic reaction in the presence of the test molecule (for a final concentration range of from 0.17 nM to 10 μM, 3% DMSO final concentration) in 10 mM MOPS buffer, pH 7.4, 1 mM DTT, 0.01% Tween 20. The reaction is initiated with the substrate solution to obtain final concentrations of 1 μg/ml poly-(GAT), 10 μM ATP and 5 mM MgCl₂. After incubation for 10 min at ambient temperature, the reaction is stopped with a 30 μl mix so as to obtain a final solution of 50 mM Hepes, pH 7.5, 500 mM potassium fluoride, 0.1% BSA and 133 mM EDTA in the presence of 80 ng of streptavidin 61 SAXLB Cis-Bio Int. and 18 ng of anti-phosphotyrosine Mab PT66-Europium Cryptate per well. After incubation for 2 hours at ambient temperature, the reading is taken at 2 wavelengths, 620 nm and 665 nm, on a reader for the TRACE/HTRF technique, and the % inhibition is calculated from the 665/620 ratios.

The results obtained with this test A for the products of formula (I) as examples in the experimental section are such that the IC50 is less than 500 nM, and in particular less than 100 nM.

B) Test B: Inhibition of the Autophosphorylation of Met; ELISA Technique (pppY1230, 1234, 1235)
a) Cell lysates: MKN45 cells are seeded into 96-well plates (cell coat BD polylysine) at 20 000 cells/well in 200 μl of RPMI medium+10% FCS+1% L-glutamine. They are left to adhere for 24 hours in an incubator.

The cells are treated the day after seeding with the products at 6 concentrations, in duplicate, for 1 h. At least 3 control wells are treated with the same final amount of DMSO.

Product dilution: Stock at 10 mM in pure DMSO—range from 10 mM to 30 μM with an increment of 3 in pure DMSO—intermediate dilutions to 1/50 in culture medium and then removal of 10 μl added directly to the cells (200 μl): final range of 10 000 to 30 nM.

At the end of the incubation, the supernatant is carefully removed and rinsing is performed with 200 μl of PBS. Next, 100 μl of lysis buffer are placed directly in the wells on ice and incubated at 4° C. for 30 minutes. Lysis buffer: 10 mM Tris HCl, pH 7.4, 100 mM NaCl, 1 mM EDTA, 1 mM EGTA, 1% Triton X-100, 10% glycerol, 0.1% SDS, 0.5% deoxycholate, 20 mM NaF, 2 mM Na₃VO₄, 1 mM PMSF and cocktail of antiproteases.

The 100 μl of lysates are transferred into a V-bottomed polypropylene plate and the ELISA is performed immediately, or the plate is frozen at −80° C.

### b) PhosphoMET ELISA BioSource kit KH00281

70 μl of kit dilution buffer+30 μL of cell lysate, or 30 μl of lysis buffer for the blanks, are added to each well of the kit plate. Incubation is carried out for 2 h with gentle agitation at ambient temperature.

The wells are rinsed 4 times with 400 μl of kit washing buffer. Incubation is carried out with 100 μl of anti-phospho MET antibody for 1 hour at ambient temperature.

The wells are rinsed 4 times with 400 μl of kit washing buffer. Incubation is carried out with 100 μl of anti-rabbit HRP antibody for 30 minutes at ambient temperature (except for the wells of chromogen alone).

The wells are rinsed 4 times with 400 μl of kit washing buffer. 100 μL of chromogen are introduced and incubation is carried out for 30 minutes in the dark at ambient temperature.

The reaction is stopped with 100 μl of stop solution. The plate is read without delay at 450 nM, 0.1 second on a Wallac Victor plate reader.

### C) Test C: Measurement of Cell Proliferation by ¹⁴C-Thymidine Pulse

The cells are seeded into Cytostar 96-well plates in 180 μl for 4 hours at 37° C. and 5% CO₂: HCT116 cells in a proportion of 2500 cells per well in DMEM medium+10% foetal calf serum+1% L-glutamine, and MKN45 cells in a proportion of 7500 cells per well in RPMI medium+10% foetal calf serum+1% L-glutamine. After these 4 hours of incubation, the products are added in 10 μl as a 20-fold concentrated solution according to the dilution method mentioned for the ELISA. The products are tested at 10 concentrations in duplicate from 10 000 nM to 0.3 nM with an increment of 3.

After treatment for 72 h, 10 μl of ¹⁴C-thymidine at 10 μCi/ml are added so as to obtain 0.1 μCi per well. The ¹⁴C-thymidine incorporation is measured on a Micro-Beta machine (Perkin-Elmer) after 24 hours of pulse and 96 h of treatment.

All the steps of the assay are automated on BIOMEK 2000 or TECAN stations.

The results obtained with this test B for the products of formula (I) as examples in the experimental section are such that the IC50 is less than 10 microM, and in particular less than 1 microM.

The results obtained for the products as examples in the experimental section are given in the pharmacological results table hereinafter, as follows:

for test A, the sign + corresponds to less than 500 nM and the sign ++ corresponds to less than 100 nM;
for test B, the sign + corresponds to greater than 500 nM and the sign ++ corresponds to less than 100 nM;
for test C, the sign + corresponds to less than 10 microM and the sign ++ corresponds to less than 1 microM.

### Pharmacological Results Table:

| Example | Test A | Test B | Test C |
|---|---|---|---|
| 1 | ++ | ++ | ++ |
| 2 | ++ | + | ++ |
| 3 | ++ | ++ | ++ |
| 4 | ++ | ++ | ++ |
| 5 | ++ | ++ | ++ |
| 6 | ++ | ++ | ++ |
| 7 | ++ | ++ | ++ |
| 8 | ++ | ++ | ++ |
| 9 | ++ | ++ | ++ |
| 10 | ++ | ++ | ++ |
| 11 | ++ | ++ | ++ |
| 12 | ++ | ++ | ++ |

1. A product of formula (I): 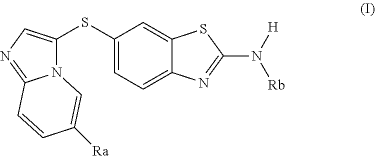
in which:
Ra represents a hydrogen atom; a halogen atom; an aryl radical; or a heteroaryl radical, these aryl and heteroaryl radicals being optionally substituted as indicated hereinafter;
Rb represents a hydrogen atom, an Rc, —COORc or —CO-Rc radical or a —CO—NRcRd radical;
where Rc represents an alkyl, cycloalkyl, heterocycloalkyl, aryl or heteroaryl radical, all these radicals being optionally substituted as indicated hereinafter;
Rd represents a hydrogen atom or an alkyl or cycloalkyl radical;
all the alkyl, cycloalkyl, heterocycloalkyl, aryl and heteroaryl radicals defined above being optionally substituted with one or more radicals chosen from halogen atoms, and the radicals: hydroxyl, alkoxy, CN, CF3, —NR1R2, —COOH, —COOalk, —CONR1R2, —NR1COR2, COR1, oxo and heterocycloalkyl, itself optionally substituted with one or more radicals chosen from halogen atoms, and hydroxyl, alkoxy, alkyl, CN, CF3, —NR3R4, COOH, —COOalk, —CONR3R4, —NR3COR4, —COR3 and oxo radicals; the alkyl and cycloalkyl radicals also being optionally substituted with an aryl or heteroaryl radical, themselves optionally substituted with one or more radicals chosen from halogen atoms and hydroxyl, alkyl, alkoxy and NR3R4 radicals;
the cycloalkyl, heterocycloalkyl, aryl or heteroaryl radicals also being optionally substituted with an alkyl radical, itself optionally substituted with one or more radicals chosen from halogen atoms and hydroxyl, alkyl, alkoxy and NR3R4 radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, chosen from halogen atoms, hydroxyl, oxo, alkoxy, NH₂, NHalk and N(alk)₂ radicals, and alkyl, phenyl, CH₂-phenyl and heteroaryl radicals, such that, in the latter radicals, the alkyl, phenyl and heteroaryl radicals are themselves optionally substituted with one or more radicals chosen from halogen atoms and the radicals: hydroxyl, alkyl and alkoxy containing from 1 to 4 carbon atoms, NH₂, NHalk and N(alk)₂;
all the alkyl (alk) and alkoxy radicals above containing from 1 to 6 carbon atoms;
racemic, enantiomeric and diastereoisomeric isomers, and pharmaceutically acceptable salts thereof.

2. The product of claim 1, in which:
Ra represents a hydrogen atom; a halogen atom; or an aryl or heteroaryl radical, these aryl and heteroaryl radicals being optionally substituted as indicated hereinafter;
Rb represents a hydrogen atom, a —CO-Rc radical or a —CO—NRcRd radical;
where Rc represents an alkyl radical or a cycloalkyl radical, both optionally substituted with one or more radicals chosen from the radicals: hydroxyl, alkoxy, NR1R2, heterocycloalkyl, aryl and heteroaryl, themselves optionally substituted as indicated hereinafter;
Rd represents a hydrogen atom or an alkyl radical;
all the alkyl, cycloalkyl, heterocycloalkyl, aryl and heteroaryl radicals defined above being optionally substituted with one or more radicals chosen from halogen atoms, and the radicals: hydroxyl, alkoxy, —NR1R2, —COOH, —COOalk, —CONR1R2, alkyl and heterocycloalkyl itself optionally substituted with one or more radicals chosen from halogen atoms, and alkyl, COOH, —COOalk and —CONR3R4 radicals;
NR1R2 being such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from hydroxyl, alkoxy, NR3R4, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R1 and R2 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
NR3R4 being such that: either, R3 and R4 being identical or different, one of R3 and R4 represents a hydrogen atom or an alkyl radical and the other of R3 and R4 represents a hydrogen atom, or a cycloalkyl radical or an alkyl radical optionally substituted with one or more radicals, which may be identical or different, chosen from hydroxyl, alkoxy, heterocycloalkyl, heteroaryl or phenyl radicals, themselves optionally substituted with one or more radicals chosen from halogen atoms and hydroxyl, alkyl, alkoxy, NH₂, NHalk and N(alk)₂ radicals; or R3 and R4 form, with the nitrogen atom to which they are attached, a cyclic radical containing from 3 to 10 ring members and optionally one or more other heteroatoms chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different, chosen from halogen atoms, hydroxyl and alkoxy radicals, and alkyl, phenyl and CH₂-phenyl radicals, in which the alkyl and phenyl radicals are themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 6 carbon atoms,
racemic, enantiomeric and diastereoisomeric isomers, and pharmaceutically acceptable salts thereof.

3. The product according to claim 1, in which:
Ra represents a hydrogen atom; a halogen atom; or a phenyl or pyrazolyl radical optionally substituted with one or more radicals chosen from halogen atoms, and the radicals: hydroxyl, alkoxy, —NR1R2, —COOH, —COOalk, —CONR1R2, alkyl and heterocycloalkyl, itself optionally substituted with one or more radicals chosen from halogen atoms, and alkyl, COOH, —COOalk and —CONR3R4 radicals;
Rb represents a hydrogen atom, a —CO-Rc radical or a —CO—NRcRd radical;
where Rc represents an alkyl or cycloalkyl radical, both optionally substituted with one or more radicals chosen from the radicals: hydroxyl, alkoxy, NR1R2 and phenyl, itself optionally substituted with one or more radicals chosen from halogen atoms, and hydroxyl, alkoxy, alkyl, NH₂, NHalk and N(alk)₂ radicals;
Rd represents a hydrogen atom or an alkyl radical;
NR1R2 is such that: either, R1 and R2 being identical or different, one of R1 and R2 represents a hydrogen atom or an alkyl radical and the other of R1 and R2 represents a hydrogen atom, or a cycloalkyl radical or an alkyl radical optionally substituted with one or more radicals, which may be identical or different, chosen from the radicals: hydroxyl, alkoxy, NR3R4, or phenyl, itself optionally substituted with one or more radicals chosen from halogen atoms and hydroxyl, alkyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, this radical, including the possible NH that it contains, being optionally substituted;
NR3R4 being such that: either R3 and R4, which may be identical or different, represent a hydrogen atom or an alkyl radical optionally substituted with one or more radicals, which may be identical or different, chosen from hydroxyl or alkoxy radicals; or R3 and R4 form, with the nitrogen atom to which they are attached, a cyclic radical containing from 4 to 7 ring members and optionally another heteroatom chosen from O, S, N and NH, this radical, including the possible NH that it contains, being optionally substituted;
the cyclic radicals that R1 and R2 or R3 and R4, respectively, can form, with the nitrogen atom to which they are attached, being optionally substituted with one or more radicals, which may be identical or different;
all the alkyl (alk) or alkoxy radicals above containing from 1 to 4 carbon atoms;
racemic, enantiomeric and diastereoisomeric isomers, and pharmaceutically acceptable salts thereof.

4. The product according to claim 1, in which:
Ra represents a hydrogen atom; a halogen atom; or a phenyl or pyrazolyl radical optionally substituted with one or more radicals chosen from halogen atoms and the radicals: alkyl and heterocycloalkyl, itself optionally substituted with one or more radicals chosen from halogen atoms and alkyl and —COOalk radicals;
Rb represents a hydrogen atom, a —CO-Rc radical or a —CO—NRcRd radical;
where Rc represents an alkyl or cycloalkyl radical optionally substituted with one or more radicals chosen from hydroxyl, alkoxy and NR1R2 radicals;
Rd represents a hydrogen atom;
NR1R2 being such that: either R1 and R2, which may be identical or different, represent a hydrogen atom or an alkyl radical optionally substituted with one or more radicals, which may be identical or different, chosen from hydroxyl, alkoxy, NH₂, NHalk and N(alk)₂ radicals; or R1 and R2 form, with the nitrogen atom to which they are attached, a cyclic radical containing from 4 to 7 ring members and optionally another heteroatom chosen from O, S, N and NH, optionally substituted with an alkyl, phenyl or —CH₂-phenyl radical, the latter radicals being themselves optionally substituted with one or more radicals, which may be identical or different, chosen from halogen atoms and alkyl, hydroxyl, alkoxy, NH₂, NHalk and N(alk)₂ radicals;
all the alkyl (alk) or alkoxy radicals above containing from 1 to 4 carbon atoms,
racemic, enantiomeric and diastereoisomeric isomers, and pharmaceutically acceptable salts thereof.

5. The product according to claim 1, in which:
Ra represents a hydrogen atom or a phenyl or pyrazolyl radical optionally substituted with one or more radicals chosen from halogen atoms and the radicals: alkyl and piperidyl, itself optionally substituted with —COOalk;
Rb represents a hydrogen atom, a —CO-Rc radical or a —CO—NRcRd radical;
where Rc represents a cyclopropyl radical or an alkyl radical optionally substituted with an alkoxy or NR1R2 radical;
Rd represents a hydrogen atom,
NR1R2 being such that: either R1 and R2, which may be identical or different, represent a hydrogen atom or an alkyl radical; or R1 and R2 form, with the nitrogen atom to which they are attached, a morpholinyl radical;
the alkyl or alkoxy radicals above containing from 1 to 4 carbon atoms;
racemic, enantiomeric and diastereoisomeric isomers, and pharmaceutically acceptable salts thereof.

6. The product according to claim 1, corresponding to the following formulae:
N-{[6-(imidazo[1,2-a]pyridin-3-yl)sulphanyl]-1,3-benzothiazol-2-yl}cyclopropanecarboxamide
6-{[6-(4-fluorophenyl)imidazo[1,2-a]pyridin-3-yl]sulphanyl}-1,3-benzothiazol-2-amine
N-(6-{[6-(4-fluorophenyl)imidazo[1,2-a]pyridin-3-yl]sulphanyl}-1,3-benzothiazol-2-yl)cyclopropanecarboxamide
N-(6-{[6-(4-fluorophenyl)imidazo[1,2-a]pyridin-3-yl]sulphanyl}-1,3-benzothiazol-2-yl)acetamide
1-(6-{[6-(4-fluorophenyl)imidazo[1,2-a]pyridin-3-yl]sulphanyl}-1,3-benzothiazol-2-yl)-3-(2-methoxyethyl)urea
1-(6-{[6-(4-fluorophenyl)imidazo[1,2-a]pyridin-3-yl]sulphanyl}-1,3-benzothiazol-2-yl)-3-[2-(morpholin-4-yl)ethyl]urea
N-(6-{[6-(1-methyl-1H-pyrazol-4-yl)imidazo[1,2-a]pyridin-3-yl]sulphanyl}-1,3-benzothiazol-2-yl)cyclopropanecarboxamide
N-(6-{[6-(1H-pyrazol-4-yl)imidazo[1,2-a]pyridin-3-yl]sulphanyl}-1,3-benzothiazol-2-yl)cyclopropanecarboxamide
N-(6-{[6-(3-fluorophenyl)imidazo[1,2-a]pyridin-3-yl]sulphanyl}-1,3-benzothiazol-2-yl)cyclopropanecarboxamide
N-(6-{[6-((3-fluoro-4-methyl)phenyl)imidazo[1,2-a]pyridin-3-yl]sulphanyl}-1,3-benzothiazol-2-yl)cyclopropanecarboxamide
tert-butyl 4-{4-[3-({2-[(cyclopropylcarbonyl)amino]-1,3-benzothiazol-6-yl}sulphanyl)imidazo[1,2-a]pyridin-6-yl]-1H-pyrazol-1-yl}piperidine-1-carboxylate
N-[6-({6-[1-(piperidin-4-yl)1H-pyrazol-4-yl]imidazo[1,2-a]pyridin-3-yl}sulphanyl)-1,3-benzothiazol-2-yl]cyclopropanecarboxamide
and pharmaceutically acceptable salts thereof.

7. A process for preparing the product according to claim 1, according to scheme 1 as defined hereinafter: 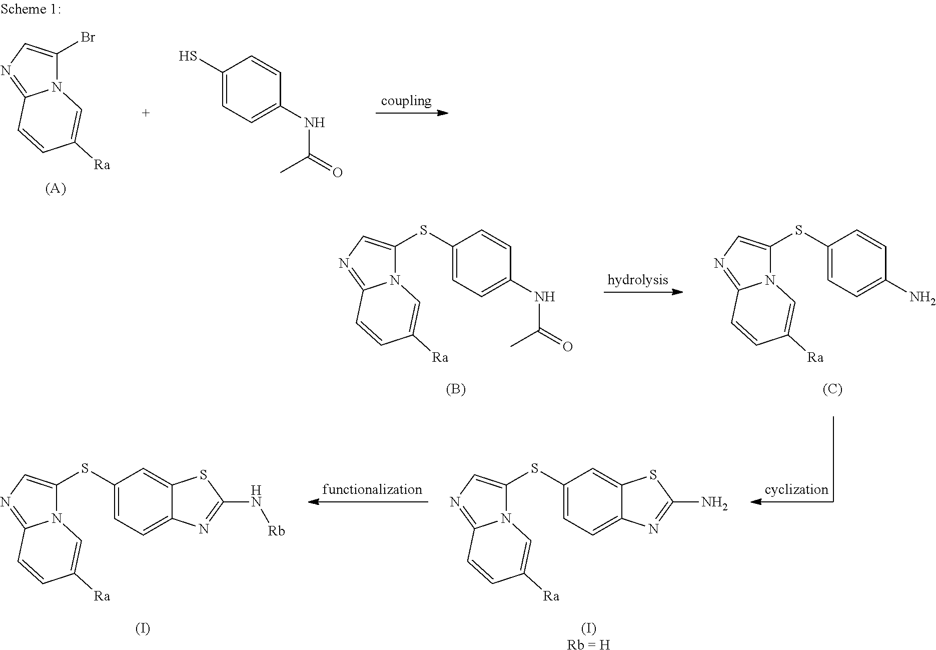
in which the substituents Ra and Rb have the meanings indicated in claim 1.

8. A process for preparing the product according to claim 1 any according to scheme 2 as defined hereinafter: 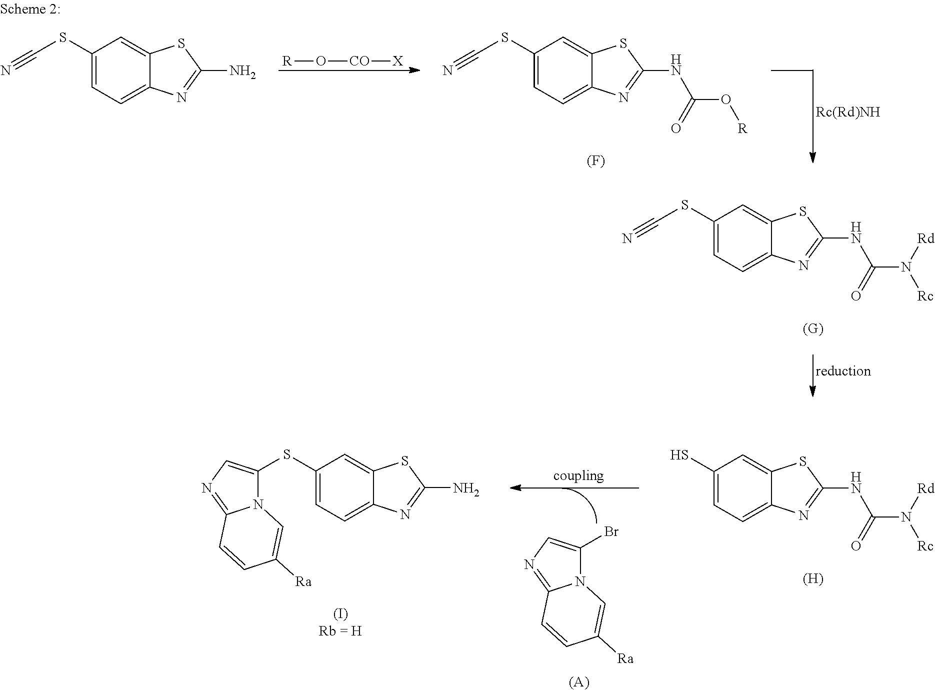
in which the substituents Ra, Rc and Rd have the meanings indicated in any one of claims 1 to 6.

9. A process for preparing the product according to claim 1, according to scheme 3 as defined hereinafter: 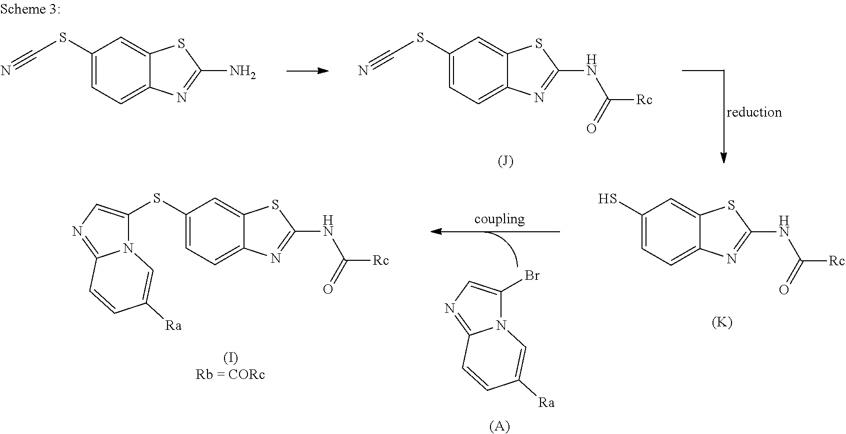
in which the substituents Ra, Rc and Rd have the meanings indicated in claim 1.

10. A pharmaceutical composition comprising the product of claim 1, and pharmaceutically acceptable salts thereof.

11. A pharmaceutical composition comprising the product of claim 6, and pharmaceutically acceptable salts thereof.

12. A pharmaceutical composition containing, as active ingredient, at least one product according to claim 1, or a pharmaceutically acceptable salt of said product or a prodrug of said product, and a pharmaceutically acceptable carrier.

13. A method of inhibiting the activity of the MET protein kinase and mutant forms thereof in a patient in need thereof comprising administering to said patient a therapeutically effective amount of the product according to claim 1, or pharmaceutically acceptable salts thereof.

14. The method of claim 13, wherein said protein kinase is in a cell culture.

15. A method of treating or preventing a disease in a patient in need thereof comprising administering to said patient a therapeutically effective amount of the product according to claim 1, wherein said disease is chosen from the following group: blood vessel proliferation disorders, fibrotic disorders, ‘mesangial’ cell proliferation disorders, metabolic disorders, allergies, asthma, thrombosis, nervous system diseases, retinopathy, psoriasis, rheumatoid arthritis, diabetes, muscle degeneration and cancers.

16. A method of treating cancers in a patient in need thereof comprising administering to said patient a therapeutically effective amount of the product according to claim 1.

17. The method according to claim 16, wherein solid or liquid tumours are treated.

18. The method according to claim 16, wherein said cancers are resistant to cytotoxic agents.

19. The method according to claim 16, comprising treating of primary tumours and/or metastases, in particular in gastric, hepatic, renal, ovarian, colon, prostate and lung (NSCLC and SCLC) cancers, glioblastomas, thyroid, bladder or breast cancers, in melanoma, in lymphoid or myeloid haematopoietic tumours, in sarcomas, in brain, larynx or lymphatic system cancers, bone cancers and pancreatic cancers.

20. (canceled)

21. (canceled)

22. The product according to claim 1, wherein said product is a kinase inhibitor inhibitors.

23. The product according to claim 1, wherein said product is a MET inhibitor.

24. A product having one of the following formulas: 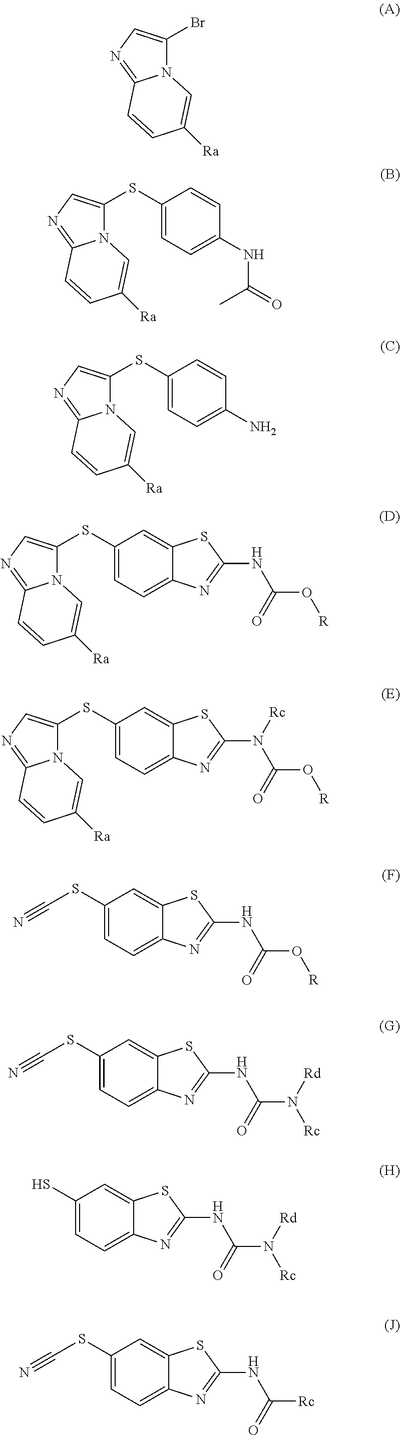 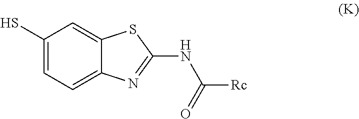
in which Ra, Rb, Rc and Rd have the definitions indicated in claim 1, and R represents a t-butyl or phenyl radical.
